SEQUENCE LISTING (75 sequences)

SEQ ID NO: 1 - protein, 108 aa, synthetic construct
SYVLTQLPSVSVTLRQTARITCGGDSIGSKNVYWYQQKLGQAPVLIIYDDSSRPSGIPERFSGANSGNTATLTISGALAEDEADYYCSTYDSLLDVGVFGGGTQLTVL

SEQ ID NO: 2 - protein, 121 aa, synthetic construct
EVQLVESGGDLVKPGGTLRLSCVASGFTFSSYDMSWVRQSPGKGLQWVAVIWNEGSSTYYADAVKGRFTISRDNAKNTLYLQMNSLRAEDTAVYYCARGRQPRLVGTLDLWGQGTLVTVSS

SEQ ID NO: 3 - protein, 11 aa, synthetic construct
GGDSIGSKNVY

SEQ ID NO: 4 - protein, 7 aa, synthetic construct
DDSSRPS

SEQ ID NO: 5 - protein, 11 aa, synthetic construct
STYDSLLDVGV

SEQ ID NO: 6 - protein, 5 aa, synthetic construct
SYDMS

SEQ ID NO: 7 - protein, 17 aa, synthetic construct
VIWNEGSSTYYADAVKG

SEQ ID NO: 8 - protein, 12 aa, synthetic construct
GRQPRLVGTLDL

SEQ ID NO: 9 - protein, 11 aa, synthetic construct
QTYDSLVDVGV

SEQ ID NO: 10 - protein, 11 aa, synthetic construct
QTYDTLKSVGV

SEQ ID NO: 11 - protein, 11 aa, synthetic construct
QTYDTITSVGV

SEQ ID NO: 12 - protein, 11 aa, synthetic construct
QTYDSLLGVGV

SEQ ID NO: 13 - protein, 11 aa, synthetic construct
QTYDHLLTVGV

SEQ ID NO: 14 - protein, 11 aa, synthetic construct
QTYDSLLTVGV

SEQ ID NO: 15 - protein, 11 aa, synthetic construct
QTYDSLLSVGV

SEQ ID NO: 16 - protein, 11 aa, synthetic construct
QSYDSLLSVGV

SEQ ID NO: 17 - protein, 11 aa, synthetic construct
QSYDSILSVGV

SEQ ID NO: 18 - protein, 11 aa, synthetic construct
  VARIANT 1: note = X = S or Q
  VARIANT 2: note = X = T or S
  VARIANT 5: note = X = S, T or H
  VARIANT 6: note = X = L or I
  VARIANT 7: note = X = L, K, T or V
  VARIANT 8: note = X = S, T, D or G
XXYDXXXXVGV

SEQ ID NO: 19 - protein, 17 aa, synthetic construct
VIWNTGSHAYYADAVKG

SEQ ID NO: 20 - protein, 17 aa, synthetic construct
WIWNESSKAAYTEAVKG

SEQ ID NO: 21 - protein, 17 aa, synthetic construct
WIWNTGSTTRYTDAVKG

SEQ ID NO: 22 - protein, 17 aa, synthetic construct
WIWNEGSTTAYAEAVKG

SEQ ID NO: 23 - protein, 17 aa, synthetic construct
WIWNSGSTTSYANAVKG

SEQ ID NO: 24 - protein, 17 aa, synthetic construct
VIWNGGSNTYYSEAVKG

SEQ ID NO: 25 - protein, 17 aa, synthetic construct
YIWNEGSGTFYTDAVKG

SEQ ID NO: 26 - protein, 17 aa, synthetic construct
WIWNEGEMTSYTEAVKG

SEQ ID NO: 27 - protein, 17 aa, synthetic construct
YIWNEGSMTYYTEAVKG

SEQ ID NO: 28 - protein, 17 aa, synthetic construct
VIWNSGGGTYYTDAVKG

SEQ ID NO: 29 - protein, 17 aa, synthetic construct
VIWNSGSSAYYADAVKG

SEQ ID NO: 30 - protein, 17 aa, synthetic construct
  VARIANT 1: note = X = W, V or Y
  VARIANT 5: note = X = E, T, S or G
  VARIANT 6: note = X = G or S
  VARIANT 7: note = X = S, E or G
  VARIANT 8: note = X = T, K, N, G, M, G, H or S
  VARIANT 9: note = X = T or A
  VARIANT 10: note = X = Y, A, R, S or F
  VARIANT 12: note = X = T, A or S
  VARIANT 13: note = X = D, E or N
XIWNXXXXXXYXXAVKG

SEQ ID NO: 31 - protein, 22 aa, synthetic construct
SYVLTQLPSVSVTLRQTARITC

SEQ ID NO: 32 - protein, 15 aa, synthetic construct
WYQQKLGQAPVLIIY

SEQ ID NO: 33 - protein, 32 aa, synthetic construct
GIPERFSGANSGNTATLTISGALAEDEADYYC

SEQ ID NO: 34 - protein, 10 aa, synthetic construct
FGGGTQLTVL

SEQ ID NO: 35 - protein, 25 aa, synthetic construct
EVQLVESGGDLVKPGGTLRLSCVAS

SEQ ID NO: 36 - protein, 14 aa, synthetic construct
WVRQSPGKGLQWVA

SEQ ID NO: 37 - protein, 32 aa, synthetic construct
RFTISRDNAKNTLYLQMNSLRAEDTAVYYCAR

SEQ ID NO: 38 - protein, 11 aa, synthetic construct
WGQGTLVTVSS

SEQ ID NO: 39 - protein, 108 aa, synthetic construct
SYVLTQLPSVSVTLRQTARITCGGDSIGSKNVYWYQQKLGQAPVLIIYDDSSRPSGIPERFSGANSGNTATLTISGALAEDEADYYCQTYDSLVDVGVFGGGTQLTVL

SEQ ID NO: 40 - protein, 121 aa, synthetic construct
EVQLVESGGDLVKPGGTLRLSCVASGFTFSSYDMSWVRQSPGKGLQWVAVIWNTGSHAYYADAVKGRFTISRDNAKNTLYLQMNSLRAEDTAVYYCARGRQPRLVGTLDLWGQGTLVTVSS

SEQ ID NO: 41 - protein, 108 aa, synthetic construct
SYVLTQLPSVSVTLRQTARITCGGDSIGSKNVYWYQQKLGQAPVLIIYDDSSRPSGIPERFSGANSGNTATLTISGALAEDEADYYCQSYAMPLTGTVFGGGTQLTVL

SEQ ID NO: 42 - protein, 119 aa, synthetic construct
EVQLVESGGDLVKPAGSLRLSCVASGFTFSSYSMSWVRQAPEKGLQLVAGISSGGSSTYYTDAVKGRFTISRDNAKNTVYLQMNSLRAEDTAMYYCARGQGGRSSFDYWGQGTLVTVSS

SEQ ID NO: 43 - protein, 11 aa, synthetic construct
QSYAMPLTGTV

SEQ ID NO: 44 - protein, 5 aa, synthetic construct
SYSMS

SEQ ID NO: 45 - protein, 17 aa, synthetic construct
WIDAYAGVTSYANAVKG

SEQ ID NO: 46 - protein, 10 aa, synthetic construct
GQGGRSSFDY

SEQ ID NO: 47 - protein, 11 aa, synthetic construct
QSYDIPLTGTV

SEQ ID NO: 48 - protein, 11 aa, synthetic construct
QSYDLPLTGTV

SEQ ID NO: 49 - protein, 11 aa, synthetic construct
  VARIANT 4: note = X = D or A
  VARIANT 5: note = X = M, I or L
QSYXXPLTGTV

SEQ ID NO: 50 - protein, 17 aa, synthetic construct
WIDATGSHTIHAEAVKG

SEQ ID NO: 51 - protein, 17 aa, synthetic construct
SIWSYGDNPIYADAVKG

SEQ ID NO: 52 - protein, 17 aa, synthetic construct
WIDAYAGVTSYANAVKG

SEQ ID NO: 53 - protein, 17 aa, synthetic construct
GITSYGSSTYYSEAVKG

SEQ ID NO: 54 - protein, 17 aa, synthetic construct
SIWSSGSGTYYTDAVKG

SEQ ID NO: 55 - protein, 17 aa, synthetic construct
GINWEGGYTYYTDAVKG

SEQ ID NO: 56 - protein, 17 aa, synthetic construct
WIDYSGSSTSYAEAVKG

SEQ ID NO: 57 - protein, 17 aa, synthetic construct
SINSSGSHITYADAVKG

SEQ ID NO: 58 - protein, 17 aa, synthetic construct
WIDYSGTRTYYADAVKG

SEQ ID NO: 59 - protein, 17 aa, synthetic construct
SINSGSSYTSYADAVKG

SEQ ID NO: 60 - protein, 17 aa, synthetic construct
  VARIANT 1: note = X = G, S or W
  VARIANT 3: note = X = D, T, N or W
  VARIANT 4: note = X = Y, A, S or W
  VARIANT 5: note = X = S, T, Y, E or G
  VARIANT 6: note = X = G, A or S
  VARIANT 7: note = X = S, T, G or D
  VARIANT 8: note = X = H, Y, S, R, V, G or N
  VARIANT 9: note = X = T, I or P
  VARIANT 10: note = X = Y, S, I or T
  VARIANT 11: note = X = Y or H
  VARIANT 12: note = X = A, T or S
  VARIANT 13: note = X = D, E or N
XIXXXXXXXXXXXAVGK

SEQ ID NO: 61 - protein, 30 aa, synthetic construct
EVQLVESGGDLVKPAGSLRLSCVASGFTFS

SEQ ID NO: 62 - protein, 14 aa, synthetic construct
WVRQAPEKGLQLVA

SEQ ID NO: 63 - protein, 32 aa, synthetic construct
RFTISRDNAKNTVYLQMNSLRAEDTAMYYCAR

SEQ ID NO: 64 - protein, 11 aa, synthetic construct
WGQGTLVTVSS

SEQ ID NO: 65 - protein, 108 aa, synthetic construct
SYVLTQLPSVSVTLRQTARITCGGDSIGSKNVYWYQQKLGQAPVLIIYDDSSRPSGIPERFSGANSGNTATLTISGALAEDEADYYCQSYDIPLTGTVFGGGTQLTVL

SEQ ID NO: 66 - protein, 119 aa, synthetic construct
EVQLVESGGDLVKPAGSLRLSCVASGFTFSSYSMSWVRQAPEKGLQLVAWIDAYAGVTSYANAVKGRFTISRDNAKNTVYLQMNSLRAEDTAMYYCARGQGGRSSFDYWGQGTLVTVSS

SEQ ID NO: 67 - protein, 271 aa, Canis sp.
FTITVSKDLYVVEYGGNVTMECKFPVEKQLNLFALIVYWEMEDKKIIQFVNGKEDLKVQHSSYSQRAQLLKDQLFLGKAALQITDVRLQDAGVYCCLIGYGGADYKRITLKVHAPYRNISQRISVDPVTSEHELMCQAEGYPEAEVIWTSSDHRVLSGKTTITNSNREEKLFNVTSTLNINATANEIFYCTFQRSGPEENNTAELVIPERLPVPASERTHFMILGPFLLLLGVVLAVTFCLKKHGRMMDVEKCCTRDRNSKKRNDIQFEET

SEQ ID NO: 68 - protein, 331 aa, Canis sp.
ASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 69 - protein, 335 aa, Canis sp.
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 70 - protein, 333 aa, Canis sp.
ASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 71 - protein, 331 aa, Canis sp.
ASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFYPPEIDVEWQSNGQPEPESKYHTTAPQLDEDGSYFLYSKLSVDKSRWQQGDTFTCAVMHEALQNHYTDLSLSHSPGK

SEQ ID NO: 72 - protein, 234 aa, synthetic construct
MVLQTQVFISLLLWISGAYASYVLTQLPSVSVTLRQTARITCGGDSIGSKNVYWYQQKLGQAPVLIIYDDSSRPSGIPERFSGANSGNTATLTISGALAEDEADYYCQTYDSLVDVGVFGGGTQLTVLGQPKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECS

SEQ ID NO: 73 - protein, 475 aa, synthetic construct
MKHLWFFLLLVAAPRWVLSEVQLVESGGDLVKPGGTLRLSCVASGFTFSSYDMSWVRQSPGKGLQWVAVIWNTGSHAYYADAVKGRFTISRDNAKNTLYLQMNSLRAEDTAVYYCARGRQPRLVGTLDLWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSGIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 74 - protein, 234 aa, synthetic construct
MVLQTQVFISLLLWISGAYASYVLTQLPSVSVTLRQTARITCGGDSIGSKNVYWYQQKLGQAPVLIIYDDSSRPSGIPERFSGANSGNTATLTISGALAEDEADYYCQSYDIPLTGTVFGGGTQLTVLGQPKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECS

SEQ ID NO: 75 - protein, 473 aa, synthetic construct
MKHLWFFLLLVAAPRWVLSEVQLVESGGDLVKPAGSLRLSCVASGFTFSSYSMSWVRQAPEKGLQLVAWIDAYAGVTSYANAVKGRFTISRDNAKNTVYLQMNSLRAEDTAMYYCARGQGGRSSFDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSGIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

(19) United States
(12) Patent Application Publication
(10) Pub. No.: US 2025/0011431 A1
(43) Pub. Date: Jan. 9, 2025

(54) ANTI-CANINE PD-L1 ANTIBODIES

(71) Applicant: adivo GmbH, Puchheim (DE)

(72) Inventors: Philipp Baer, Puchheim (DE); Stephanie Patzelt, Puchheim (DE); Felix Funck, Puchheim (DE)

(21) Appl. No.: 18/617,442

(22) Filed: Mar. 26, 2024

(30) Foreign Application Priority Data
Mar. 27, 2023 (EP) EP23164436.0

(51) Int. Cl. C07K 16/28; A61K 39/00; A61P 35/00

(52) U.S. Cl. CPC C07K 16/2827; A61P 35/00; A61K 2039/505; C07K 2317/20; C07K 2317/52; C07K 2317/565; C07K 2317/567; C07K 2317/76; C07K 2317/92

(57) ABSTRACT

The present invention relates to anti-canine PD-L1 antibodies. The antibodies are preferably fuly-canine antibodies. The present invention further relates to epitopes of canine PD-L1 that are bound by these antibodies and which inhibit binding of canine PD-1 to PD-L1. The invention further relates to use of the antibodies of the present invention in the treatment of dogs, including cancer treatment.

### CROSS REFERENCE TO RELATED APPLICATIONS

This application claims priority from EP 23164436.0 filed on Mar. 27, 2023, which is incorporated by reference in its entirety.

### FIELD OF THE INVENTION

The present invention relates to anti-canine PD-L1 antibodies. The antibodies are preferably fully canine antibodies. The present invention further relates to epitopes of canine PD-L1 that are bound by these antibodies, and which inhibit binding of canine PD-1 to PD-L1. The invention further relates to use of the antibodies of the present invention in the treatment of dogs, including cancer treatment.

### REFERENCE TO AN ELECTRONIC SEQUENCE LISTING

The contents of the electronic sequence listing (ZP000471A_ST26_Sequence_Listing_2024-03-22.xml; size: 79 kilobytes; and Production Date: Mar. 22, 2024) is herein incorporated by reference in its entirety.

### BACKGROUND

In human medicine, many advancements have been made in immunotherapeutic management of cancer, especially malignant melanoma with the introduction of immune checkpoint inhibitors (ICIs). Ipilimumab, an anti-CTLA-4 monoclonal antibody, was the first approved ICI that showed efficacy in human advanced/metastatic melanoma. Subsequently, nivolumab and pembrolizumab, both of which are anti-PD-1 antibodies, were approved for advanced melanoma treatment. In total, since May 2006 six anti-PD1/PD-L1 specific ICIs were approved by the FDA and/or EMA for various cancer indications: pembrolizumab (anti-PD1, IgG4k), nivolumab (anti-PD1, IgG4), cemiplimab (anti-PD1, IgG4), atezolizumab (anti-PD-L1, IgG1, silent Fc format), avelumab (anti-PD-L1, IgG1, wild-type Fc format) and durvalumab (anti-PD-L1, IgG1, silent Fc format) (Ai et al. 2020).

ICIs are a true breakthrough in human melanoma treatment, strikingly improving the prognosis of responder patients. Hence, veterinary medicine is now shifting attention to the use of ICIs as a potentially effective systemic treatment also for tumor-bearing dogs. However, in dogs, the experience is limited. The expression of Cytotoxic T lymphocyte associated protein 4 (CTLA-4), Programmed death-1 (PD-1) and of PD-1 ligand-1 (PD-L1) on canine immune cells and/or cancer cells has already been investigated and reported (Igase et al. 2020; Maekawa et al. 2017; Mason et al. 2021).

PD-1 is a 55 kDa type I transmembrane glycoprotein containing an extracellular Ig Variable-type (V-type) domain that binds its ligands and a cytoplasmic tail that binds signalling molecules. PD-1 is an inhibitor of both adaptive and innate immune responses, and is expressed on activated T cells, natural killer (NK) cells, B lymphocytes, macrophages, dendritic cells (DCs) and monocytes. Importantly, PD-1 is highly expressed on tumor-specific T cells (Ahmadzadeh et al. 2009). PD-1 plays two opposing roles, being both beneficial and harmful. It plays a key role in reducing the regulation of ineffective or damaging immune responses. Thus PD-1 maintains immune tolerance by suppressing harmful responses against self-proteins. On the other hand, PD-1 causes the expansion of malignant cells by impairing the protective immune response. Suppression of the immune response is achieved by binding of PD-L1, a ligand of PD-1, to PD-1.

PD-L1 is a 33 kDa type I transmembrane glycoprotein that contains both IgV-and IgC-like domains in the extracellular domain along with short cytoplasmic regions with no known signalling motifs (Sanmamed und Chen 2014). PD-L1 is usually expressed by macrophages, some activated T cells and B cells, DCs and some epithelial cells, particularly under inflammatory conditions (Han et al. 2020). Moreover, PD-L1 is overexpressed in a variety of tumors, where it binds to PD-1, inhibits the proliferation of PD-1-positive cells, and participates in the immune evasion of tumors leading to treatment failure (Ohaegbulam et al. 2015). Such tumor tissues include cancers of the lung, liver, ovary, cervix, skin, colon, glioma, bladder, breast, kidney, esophagus, stomach, oral squamous cell, urothelial cell, and pancreas, as well as tumors of the head and neck.

PD-L1 on the surface of tumor cells can be upregulated by interferon gamma (IFN-γ) produced by activated T cells (Tang et al. 2018) and NK cells (Bellucci et al. 2015). Binding of PD-L1 on tumor cells to PD-1 on immune cells can diminish T cell-mediated immune surveillance, resulting in an absence of immunoreaction against the tumor and even to apoptosis of T cells (Iwai et al. 2017).

In summary, aberrant PD-L1 expression has been reported in many human cancers and is considered an immune escape mechanism for cancers. Importantly, PD-L1 expression has also been demonstrated in a variety of canine cancers, particularly in oral malignant melanoma (OMM) (36/40 patients; (Maekawa et al. 2016). Other PD-L1 positive cancers include, osteosarcoma, hemangiosarcoma, mast cell tumor, mammary adenocarcinoma, and prostate adenocarcinoma (Takeuchi et al. 2020; Maekawa et al. 2014; Cascio et al. 2021; Ariyarathna et al. 2020; Hartley et al. 2017; Maekawa et al. 2016). Only recently, a higher expression of PD-L1 by neoplastic lymphocytes compared to normal B-cells was demonstrated by flow-cytometry canine B-cell lymphoma (Hartley et al. 2018). Increased PD-L1 expression is associated with a higher risk of progression and lymphoma-related death regardless of treatment (Aresu et al. 2021). This result is in line with recent evidence in humans, suggesting that upregulation of PD-L1 in tumor cells allows tumors to elude the host's immune system and increase chemoresistance. PD-1 expression is high on tumor-infiltrating lymphocytes obtained from oral melanoma, showing that lymphocytes in this cancer type might have been functionally exhausted (Maekawa et al. 2016). Similar results were shown in another study on canine melanoma cell lines and tumor infiltrating macrophages that upregulated PD-L1 expression upon exposure to interferon-y, suggesting an important mechanism of tumor-mediated T cell suppression (Hartley et al. 2017).

A few attempts have been made to design therapeutic antibodies candidates against canine ICIs, yet the clinical experience is in its infancy. Chimeric rat-dog anti-PD-L1 (Maekawa et al. 2017) and “caninized” anti-CTLA-4 (Mason et al. 2021) and anti-PD-1 (Igase et al. 2020) monoclonal antibodies (mAbs) have been developed.

Maekawa and colleagues demonstrated that a canine-chimeric PD-L1 monoclonal antibody (referred to as c4G12) enhanced cytokine production and proliferation of dog peripheral blood mononuclear cells (Maekawa et al. 2017). More importantly, in a pilot clinical study using c4G12, anti-tumor responses were observed in one of seven dogs with oral malignant melanoma and one of two dogs with undifferentiated sarcoma (Maekawa et al. 2017). Anti-tumor response and increased overall survival time following c4G12 treatment was also reported in a follow-up study with 29 dogs with diagnosed primary OMM and confirmed lung metastases (Maekawa et al. 2021).

It is noteworthy that this canine-chimerized antibody features variable light (VL) and heavy chains (VH) from rat origin, representing a significant source for the development of anti-drug antibodies (ADAs). The authors did not comment on investigating the presence of ADAs in any of the dogs treated. However, ADAs may reduce the compound's pharmaceutical efficacy after repeated dosing. In addition, there is an increased risk that chimeric antibodies cause allergic reaction after administration, with common side effects including fever, chills, headache, nausea, vomiting, diarrhea, rashes or weakness.

The generation of species-specific mAbs expected to have better safety profiles is technically challenging and experience in the development of antibodies for companion animals is only gradually developing. There are only a few technological approaches available to generate therapeutic antibodies for companion animals such as dogs or cats, namely the modification of existing compounds and the use of transgenic animals. Methods to “caninize” or “felinize” antibodies have been disclosed (Gearing et al. 2016; Gearing et al. 2013). However, even subtle changes of the protein sequence of an antibody can result in significant loss of efficacy and altered biophysical properties, rendering such methods time consuming and prone to failure. More advanced technologies relate to transgenic rodents that express canine immunoglobulins (Wabl May 23, 2017). Disadvantages are the need to sacrifice animals for the initial antibody discovery process and that the immunization process is hardly controllable. In this respect, in vitro selection methods such as phage display offer huge advantages as these allow a tailored antibody selection process. Only recently, a synthetic phage display library comprising fully canine antibody fragments has been disclosed (Tiller et al. Jun. 21, 2018).

The present invention relates to fully canine antibodies against canine PD-L1 derived from a species-specific canine phage display library with high target specificity and favourable biophysical properties to be used therapeutically in a variety of canine malignancies.

The citation of any reference herein should not be construed as an admission that such reference is available as “prior art” to the instant application.

### SUMMARY OF INVENTION

The present invention relates to antibodies or antibody fragments that bind to canine Programmed Death Ligand 1 (canine PD-L1). The antibodies or antibody fragments are characterized by their complementary determining regions (CDR) or their light and/or heavy variable domains.

In a first aspects the inventors identified antibodies or antibody fragments that may comprise a light chain CDR1 (LCDR1) region according to SEQ ID No.: 3, a light chain CDR2 (LCDR2) region according to SEQ ID No.: 4, and/or a light chain CDR3 (LCDR3) region according to SEQ ID No.: 18 or SEQ ID No.: 49. Furthermore, the antibodies or antibody fragments may comprise a heavy chain CDR 1 (HCDR1) region according to SEQ ID No.: 6 or SEQ ID No.: 44, a heavy chain CDR 2 (HCDR2) region according to SEQ ID No.: 30 or SEQ ID No.: 60, and/or a heavy chain CDR 3 (HCDR3) region according to SEQ ID No.: 8 or SEQ ID No.: 45.

By maturing an antibody having a light chain variable domain according to SEQ ID No.: 1, comprising LCDR3 according to SEQ ID No.: 5 combined with a heavy chain variable domain according to SEQ ID No.: 2 comprising HCDR2 according to SEQ ID No.: 7, several high affinity antibodies comprising LCDR3 regions having an amino acid sequence selected from any one of SEQ ID No.: 9, 10, 11, 12, 13, 14, 15, 16, and 17, and a HCDR2 region having an amino acid sequence selected from any one of SEQ ID No.: 19, 20, 21, 22, 23, 24, 25, 26, 27, 28, and 29 were obtained. Based on these LCDR3 regions, a first anti-PD-L1 high affinity LCDR3 consensus sequence according to SEQ ID No.: 18, and a first anti-PD-L1 high affinity HCDR2 consensus sequence according to SEQ ID No.: 30 could be identified by the inventors.

In an alternative aspect of the invention, by maturing an antibody having a light chain variable domain according to SEQ ID No.: 41, comprising LCDR3 according to SEQ ID No.: 43 combined with a heavy chain variable domain according to SEQ ID No.: 42 comprising HCDR2 according to SEQ ID No.: 45, several high affinity antibodies comprising LCDR3 regions having an amino acid sequence selected from SEQ ID No.: 47, or 48, and a HCDR2 region having an amino acid sequence selected from any one of SEQ ID No.: 50, 51, 52, 53, 54, 55, 56, 57, 58, and 59, preferably SEQ ID No.: 19, or 52 were obtained. Based on these LCDR3 regions, a second anti-PD-L1 high affinity LCDR3 consensus sequence according to SEQ ID No.: 49, and a second anti-PD-L1 high affinity HCDR2 consensus sequence according to 60 could be identified by the inventors.

Accordingly, the antibodies or antibody fragments according to the invention may comprise a high affinity LCDR3 consensus sequence according to SEQ ID No.: 18 or SEQ ID No.: 49, and/or high affinity HCDR2 consensus sequence according to SEQ ID No.: 30 or 60.

Preferably the antibodies or antibody fragments comprise a light chain comprising a combination of the various LCDR1, LCDR2 and LCDR 3 disclosed herein, and/or a heavy chain comprising a combination of the various HCDR1, HCDR2 and HCDR 3 disclosed herein. Most preferably the antibodies or antibody fragments comprise a light chain comprising a combination of the LCDR1, LCDR2 and LCDR 3 disclosed herein with a heavy chain comprising the HCDR1, HCDR2 and HCDR 3 disclosed herein.

In a preferred embodiment the antibodies or antibody fragments comprise a variable light chain comprising a LCDR3 region according to SEQ ID No.: 18 combined with a variable heavy chain comprising a HCDR2 region according to SEQ ID No.: 30 or comprises a variable light chain comprising a LCDR3 region according to SEQ ID No.: 49 combined with a variable heavy chain comprising a HCDR2 region according to SEQ ID No.: 60.

In specific preferred embodiments, the invention provides anti-PD-L1 antibodies with a surprisingly high affinity comprising a light chain variable domain according to SEQ ID No.: 39, combined with a heavy chain variable domain according to SEQ ID No.: 40 or comprising a light chain variable domain according to SEQ ID No.: 65, combined with a heavy chain variable domain according to SEQ ID No.: 66.

The antibodies or antibody fragments preferably are fully canine, optionally a recombinant canine antibodies or antibody fragments. antibodies or antibody fragments may be used in the treatment of a disease in a subject preferably a canine subject in need thereof. The disease may for example be cancer or an inflammatory or autoimmune disease.

Notably, the fully canine antibodies according to the invention may overcome the immunogenic liabilities related to chimerized antibodies derived from non-canine species, such as rodent origin. Importantly the chimerized antibodies described in literature only display weak affinities, potentially severely limiting their use as therapeutic antibodies.

Furthermore, the invention relates to pharmaceutical compositions comprising the antibodies or antibody fragments, polynucleotides encoding the antibody or antibody fragment, vectors comprising the polynucleotide or plurality of polynucleotides disclosed herein. In a preferred embodiment the pharmaceutical composition of the invention comprises a therapeutically effective amount of the antibody of the invention further comprising a pharmaceutically acceptable carrier.

In one or more embodiments, the antibody or antibody fragments of the invention provide a method of treating a PD-L1 related disorder. In one or more embodiments the PD-L1 related disorder is cancer. In one or more embodiments the type of cancer is selected from, but not limited to: melanoma, lung cancer, bladder cancer, renal cell carcinoma, head and neck cancer, breast cancer, esophageal cancer, and lymphoma. In a preferred embodiment the PD-L1 disorder is melanoma.

In one or more aspects, the present invention provides a method of treating a subject for PD-L1-related disorder comprising administering to said subject a therapeutically effective amount of the pharmaceutical composition the present invention.

In one or more embodiments, the invention provides a host cell that produces any one or more of the antigen binding proteins of the present invention.

In one or more embodiments, the invention provides a vector comprising the any one or more of the polynucleotides that encode the antibody or antibody fragment of the present invention.

In one or more embodiments, the invention provides a host cell comprising the any one or more of the polynucleotides of the present invention.

In one or more embodiments, the invention provides a host cell comprising the vector comprising any one or more of the nucleic acids of the present invention.

In one or more embodiments, the invention provides a host cell comprising any one or more of the nucleic acids of the present invention.

In one or more aspects, the invention provides a method of producing the antigen binding protein of the invention by culturing the host cell of the invention under conditions that result in production of the antigen binding protein and subsequently isolating the antigen binding protein from the host cell or culture medium of the host cell.

In one or more aspects, the invention provides a kit comprising the antibody or antibody fragment of the invention.

### FIGURE LEGEND

FIGS. 1A-1B: Consensus sequences based on maturation of LCDR3 (FIG. 1A) and HCDR2 region (FIG. 1B) of CAN1005010.

FIGS. 2A-2B: Consensus sequences based on maturation LCDR3 (FIG. 2A) and HCDR2 region (FIG. 2B) of CAN1005001.

FIGS. 3A-3B: cPD-L1 binding and neutralization of the cPD-L1/cPD-1 interaction by CAN1005001 (FIG. 3B) and CAN1005010 (FIG. 3A)

FIGS. 4A-4C: Concentration dependent binding of CAN1005010L1 and CAN1005010H2 (FIG. 4. A), CAN1005001L1, CAN1005001H3, and CAN1005001 (FIG. 4.B), and derived IC50 values (FIG. 4.C) in cPD-L1-biotin ELISA assay.

FIGS. 5A-5B: Concentration dependent binding of CAN1005016 (FIG. 5. A) and CAN1005019 (FIG. 5. B) in cPD-L1-biotin ELISA assay.

FIGS. 6A-6B: Affinity measurement of CAN1005016 (FIG. 6. A) and CAN1005019 (FIG. 6. B) by GCI by 2BIND

FIGS. 7A-7B: cPD-L1 binding of candidates derived from cross-cloning maturated IgGs. Titration series of biotinylated CAN1005016 (FIG. 7. A) and CAN1005019 (FIG. 7. B) were added to plate-bound cPD-L1_hFc. FIGS. 8A-8C: FACS binding of CAN1005016 (FIG. 8A), CAN1005019 (FIG. 8B) and control IgG (FIG. 8C) HEK293c18 cells transfected with the target protein.

FIGS. 9A-9B: ELISA-based ligand-binding inhibition assay of CAN1005016 (FIG. 9A), CAN1005019 (FIG. 9B).

FIG. 10: Biolayer-Interferometry (BLI)-based-PD-1 binding inhibition assay of CAN1005016 antibody candidate.

FIGS. 11A-11C: Flow cytometry results of control or IFN-g treated SCC1 cells stained with CAN1005016 (FIG. 11A), CAN1005019 (FIG. 11B), or canine IgG control (FIG. 11C).

### Definitions

Unless defined otherwise, all technical and scientific terms used herein have the same meaning as commonly understood by those of ordinary skill in the area to which this invention pertains.

As used in this specification and the appended claims, the singular forms “a,” “an” and “the” include plural references unless the context clearly dictates otherwise.

“About” in the context of amount values refers to an average deviation of maximum+/−20%, preferably +/−10%, most preferably +/−5% based on the indicated value. For example, an amount of about 20 mg/ml refers to 20 mg/ml+/−6 mg/ml, preferably 20 mg/ml+/−4 mg/ml, most preferably 20 mg/ml+/−2 mg/ml. This also includes the value itself without any deviation.

All ranges set forth herein in the summary and description of the invention include all numbers or values thereabout or there between of the numbers of the range. The ranges of the invention expressly denominate and set forth all integers, decimals, and fractional values in the range. The term “about” can be used to describe a range.

The terms “antibody” or “polypeptide binder” as used herein include whole antibodies and any antigen binding fragment (i.e., “antigen-binding portion”) or single chains thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region of an IgG, IgA or IgD antibody is comprised of three domains, CH1, CH2 and CH3, whereas the heavy chain of an IgM and IgE antibody is comprised of four domains CH1, CH2, CH3, CH4. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FW). Each VH and VL is composed of three CDRs and four FWs arranged from amino-terminus to carboxy-terminus in the following order: FW1, CDR1, FW2FW2, CDR2, FW3, CDR3, FW4. A polypeptide comprising FW1, CDR1, FW2, CDR2, FW3, CDR3, FW4 of a variable region of the heavy and light chain may be referred to as a “VH or VL polypeptide”. The variable regions of the heavy and light chains contain a binding domain that interacts with an antigen. The constant regions of the antibodies may mediate the binding of the immunoglobulin to host tissues or factors, including various cells of the immune system (e.g., effector cells) via Fc-receptors and the first component (Clq) of the classical complement system.

The extent of the framework region and CDRs have been precisely defined for human antibodies (see Kabat, 1991, J. Immunol., 147, 915-920.; Chothia & Lesk, 1987, J. Mol. Biol. 196:901-917; Chothia et al., 1989, Nature 342:877-883; Al-Lazikani et al., 1997, J. Mol. Biol. 273:927-948). The framework regions of an antibody, that is, the combined framework regions of the constituent light and heavy chains, serves to position and align the CDRs, which are primarily responsible for binding to an antigen. However, although canine antibodies can in part be aligned to human antibodies, the above-mentioned numbering schemes are not ideally suited to describe amino acid positions within an antibody heavy or light chain sequence. In this invention, the following numbering scheme is used:

The antibody heavy chain is defined as VH FW1-HCDR1-FW2-HCDR2-FW3-HCDR3-FW4.

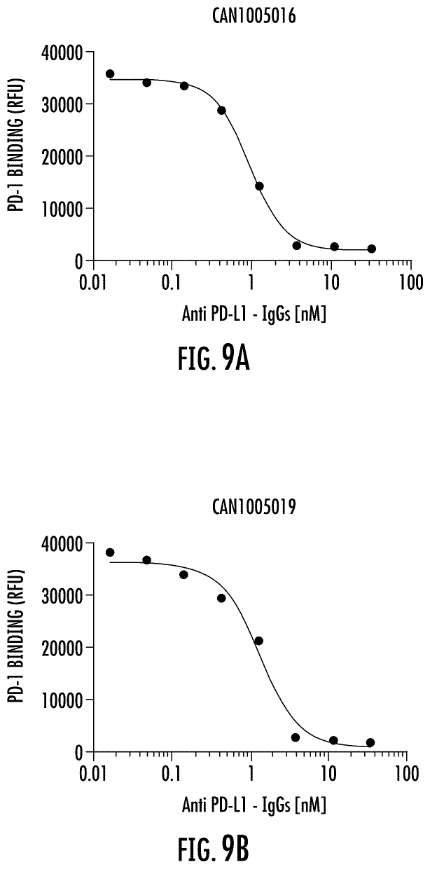

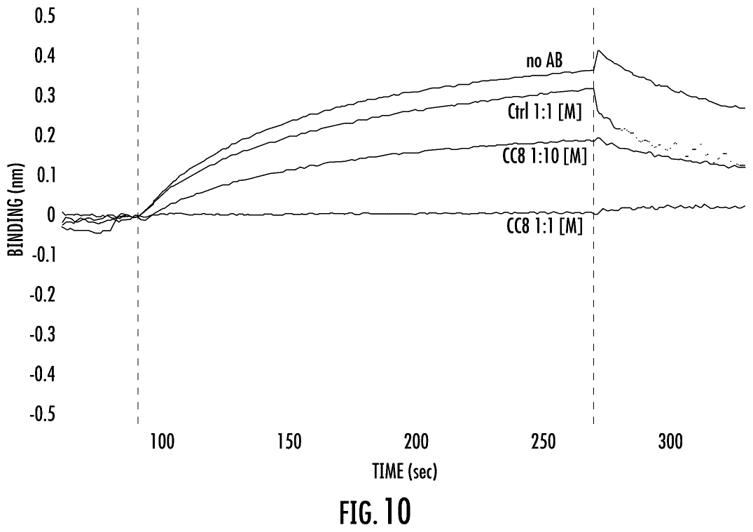

Framework 1 (FW1) is composed of 30 amino acids (X1, X2, X3, X4, X5, X6, X7, X8, X9, X10, X11, X12, X13, X14, X15, X16, X17, X18, X19, X20, X21, X22, X23, X24, X25, X26, X27, X28, X29, X30). The HCDR1 is 5 amino acids in length and defined from position X31 to X35 (X31, X32, X33, X34, X35). Framework 2 (FW2) is 14 amino acids in length and defined from position X36 to X49 (X36, X37, X38, X39, X40, X41, X42, X43, X44, X45, X46, X47, X48, X49). The HCDR2 is defined from position X50 to X65 (X50, X51, X52, X52a, X53, X54, X55, X56, X57, X58, X59, X60, X61, X62, X63, X64, X65). Framework 3 (FW3) is defined from position X66 to X94 and is 32 amino acids in length. The HCDR3 is defined from position X95 to X102 (X98, X99, X100, X100a, X100b, X100c, X100d, X100e, X100f, X100g, X101, X102,). This CDR is diverse in length, indicated by positions X100a to X100g that can harbor an amino acid or not. For clarification, if one of these positions is empty, the subsequent positions until X101 are empty as well. Framework 4 (FW4) is defined from position X103 to X113 (X103, X104, X105, X106, X107, X108, X109, X111, X112, X113). The general concept of the numbering scheme is also depicted in FIG. 9.

The antibody light chain is defined as VL FW1-LCDR1-FW2-LCDR2-FW3-LCDR3-FW4. Framework 1 (FW1) is defined from position Y1 to Y23, with one length variation at position 10, for clarification, position Y10 can be empty or having an amino acid (Y1, Y2, Y3, Y4, Y5, Y6, Y7, Y8, Y9, Y10, Y11, Y12, Y13, Y14, Y15, Y16, Y17, Y18, Y19, Y20, Y21, Y22, Y23). The LCDR1 is 11 amino acids in length and defined from position 24 to 34 (Y24, Y25, Y26, Y27, Y28, Y29, Y30, Y31, Y32, Y33, Y34). Framework 2 (FW2) defined from position 35 to 49 (Y35, Y36, Y37, Y38, Y39, Y40, Y41, Y42, Y43, Y44, Y45, Y46, Y47, Y48, Y49). The LCDR2 is 7 amino acids in length and defined from position 50 to 56, (Y50, Y51, Y52, Y53, Y44, Y55, Y56). Framework 3 (FW3) is defined from position Y57 to Y88 (Y57, Y58, Y59, Y60, Y61, Y62, Y63, Y64, Y65, Y66, Y67, Y68, Y69, Y70, Y71, Y72, Y73, Y74, Y75, Y76, Y77, Y78, Y79, Y80, Y81, Y82, Y83, Y84, Y85, Y86, Y87, Y88). The LCDR3 is defined from position Y89 to Y97 (Y89, Y90, Y91, Y92, Y93, Y94, Y95, Y95a, Y95b, Y95c, Y96, Y97). This CDR has length variation, indicated by positions Y95a to Y95c that can harbor an amino acid or not. For clarification, if one of these positions is empty, the subsequent positions until Y91 are empty as well. Framework 4 (FW4) is defined from position Y98 to Y107 (Y98, Y99, Y100, Y101, Y102, Y103, Y104, Y105, Y106, Y107).

The terms “antigen binding portion” or “fragment” of an antibody are used equivalently in the present application. These terms refer to one or more fragments of an intact antibody that retain the ability to specifically bind to a given antigen. Antigen binding functions of an antibody can be performed by fragments of an intact antibody. Examples of binding fragments encompassed within the term “antigen binding portion” of an antibody include a Fab fragment, a monovalent fragment consisting of the VL, VH, CL and CH1 domains; a F (ab) 2 fragment, a bivalent fragment comprising two Fab fragments linked by a disulfide bridge at the hinge region; an Fd fragment consisting of the VH and CH1 domains; an Fv fragment consisting of the VL and VH domains of a single arm of an antibody; a single domain antibody (dAb) fragment (Ward et al., 1989 Nature 341:544-546), which consists of a VH domain; and an isolated complementarity determining region (CDR). Preferred antigen binding portions or fragments of antibodies are Fab fragments. Furthermore, although the two domains of the Fv fragment, VL and VH, are coded for by separate genes, they can be joined, using recombinant methods, by an artificial peptide linker that enables them to be made as a single protein chain in which the VL and VH regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies include one or more “antigen binding portions” of an antibody. These antibody fragments are obtained using conventional techniques known to those of skill in the art, and the fragments are screened for utility in the same manner as are intact antibodies. Antigen binding portions can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology, 23, 9, 1126-1136). A type of single-domain antibodies are heavy chain variable domains referred to herein as “VHH” (heavy chain variable domain of heavy-chain antibody) derived from HCAb (heavy chain antibodies) found in camelid animals (such as Camelus dromedarius, Camelus bactrianus, Vicugna pacos, or Lama glama). V-NAR are heavy chain only binders derived from have heavy-chain antibodies (IgNAR, “immunoglobulin new antigen receptor′) from cartilaginous fishes. Antigen binding portions of antibodies can be grafted into scaffolds based and polypeptides such as Fibronectin type III (Fn3) (see U.S. Pat. No. 6,703,199, which describes fibronectin polypeptide monobodies). Antigen binding portions can be incorporated into single chain molecules comprising a pair of tandem Fv segments (VH—CH1-VH-CH1) which, together with complementary light chain polypeptides, form a pair of antigen binding regions (Zapata et al., 1995 Protein Eng. 8 (10) 1 057-1062; and U.S. Pat. No. 5,641,870).

The term “isolated” refers to a compound, which can be e.g., an antibody or antibody fragment, that is substantially free of other antibodies or antibody fragments having different antigenic specificities. Moreover, an isolated antibody or antibody fragment may be substantially free of other cellular material and/or chemicals. Thus, in some aspects, antibodies provided are isolated antibodies which have been separated from antibodies with a different specificity. An isolated antibody may be a monoclonal antibody. An isolated antibody may be a recombinant monoclonal antibody. An isolated antibody that specifically binds to an epitope, isoform or variant of a target may, however, have cross-reactivity to other related antigens, e.g., from other species (e.g., species homologs).

The term “fully canine antibody”, as used herein, refers to antibodies having variable regions in which both the framework and CDR regions are derived from sequences of canine origin. For example, both, the framework and CDR regions may be derived from sequences of canine origin. Furthermore, if the antibody contains a constant region, the constant region also is derived from such canine sequences, e.g., canine germline sequences, or mutated versions of canine germline sequences. The canine antibodies of the invention may include amino acid residues not encoded by canine sequences (e.g., mutations introduced by random or site-specific mutagenesis in vitro or by somatic mutation in vivo).

The terms “monoclonal antibody” or “monoclonal antibody composition” as used herein refer to a preparation of antibody molecules of single molecular composition. A monoclonal antibody composition displays a single binding specificity and affinity for a particular epitope.

The term “germline” refers to fully germline sequences and in addition to germline sequences that have been modified or engineered with minor mutations in the amino acid sequence, such as, for the purpose of removing of undesired post-translational modification (PTM) sites, of removing undesired cysteine, optimizing the antibody (e.g. affinity, half-life) or introduction of desired restriction site, or modifications that result from errors in synthesis, amplification or cloning.

As used herein, the term “affinity” refers to the strength of interaction between the polypeptide and its target at a single site. Within each site, the binding region of the polypeptide interacts through weak non-covalent forces with its target at numerous sites, the more interactions, the stronger the affinity.

The term “KD”, as used herein, refers to the dissociation constant, which is obtained from the ratio of Kd to Ka (i.e., Kd/Ka) and is expressed as a molar concentration (M). K_{D} values for antigen binding moieties like e.g., monoclonal antibodies can be determined using methods well established in the art. Methods for determining the Kp of an antigen binding moiety like e.g., a monoclonal antibody are SET (soluble equilibrium titration) or surface plasmon resonance using a biosensor system such as a Biacore® system.

A light chain variable domain described herein “combined with” a heavy chain variable domain relates to a paired light and heavy chains paired with each other. The pairing may be between different domains of one polypeptide chain comprising the VL polypeptide sequence and the VH polypeptide sequence, as for example in an scFv, or between two polypeptide chains comprising the VL polypeptide sequence and the VH polypeptide sequence, as for example in a full-length antibody or a Fab fragment.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids are identified herein according to the commonly known one-letter or three-letter amino acid code.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The terms “identical” or percent “identity,” in the context of two or more nucleic acids or polypeptide sequences, refer to two or more sequences or sub-sequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length. A “substantially identically sequence” according to the invention may thus include any of the disclosed sequences having one or two or three or four or five amino acid exchange(s), preferably one to three, more preferably one or two amino acid exchange(s). Preferably, the exchanges may be conservative amino acid exchange(s).

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the programmed parameters. Alignment for purposes of determining percent sequence identity within the present invention can be carried out in various ways well known to the person skilled in the art, for instance, using publicly available computer software such as BLAST, BLAST-2, ALIGN, or MEGALINETM (DNASTAR) software. The person skilled in the art is routinely able to determine appropriate parameters for measuring alignment, including any algorithms needed to achieve maximal alignment over the full length of sequences being compared. Unless otherwise defined the alignment is performed with the default settings of the alignment tool.

The term “recombinant host cell” (or simply “host cell”) refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “vector” refers to a polynucleotide molecule capable of transporting another polynucleotide to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors can direct the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses, and adeno-associated viruses), which serve equivalent functions.

“Cross compete” means the ability of an antibody, antibody fragment or other antigen-binding moieties to interfere with the binding of other antibodies, antibody fragments or antigen-binding moieties to a specific antigen in a standard competitive binding assay. The ability or extent to which an antibody, antibody fragment or other antigen-binding moieties can interfere with the binding of another antibody, antibody fragment or antigen-binding moieties to a specific antigen, and, therefore whether it can be said to cross-compete according to the invention, can be determined using standard competition binding assays. One suitable assay involves the use of the Biacore technology (e.g., by using the BIAcore 3000 instrument (Biacore, Uppsala, Sweden)), which can measure the extent of interactions using surface plasmon resonance technology.

### DETAILED DESCRIPTION

In a general aspect the invention relates to antibody or antibody fragment comprising a LCDR1 region according to SEQ ID No.: 3, a LCDR2 region according to SEQ ID No.: 4, and/or a LCDR3 region according to SEQ ID No.: 18 or SEQ ID No.: 49. The antibody or antibody fragment may further comprise a HCDR1 region according to SEQ ID No.: 6 or SEQ ID No.: 44, a HCDR2 region according to SEQ ID No.: 30 or SEQ ID No.: 60, and/or a HCDR3 region according to SEQ ID No.: 8 or SEQ ID No.: 46.

The antibodies and antibody fragments described herein bind specifically to canine programmed cell death 1 ligand 1 (PD-L1) which may for example be characterised by an amino acid sequence according to NCBI Reference Sequence: NP_001278901.1 or a sequence according to SEQ ID NO: 67.

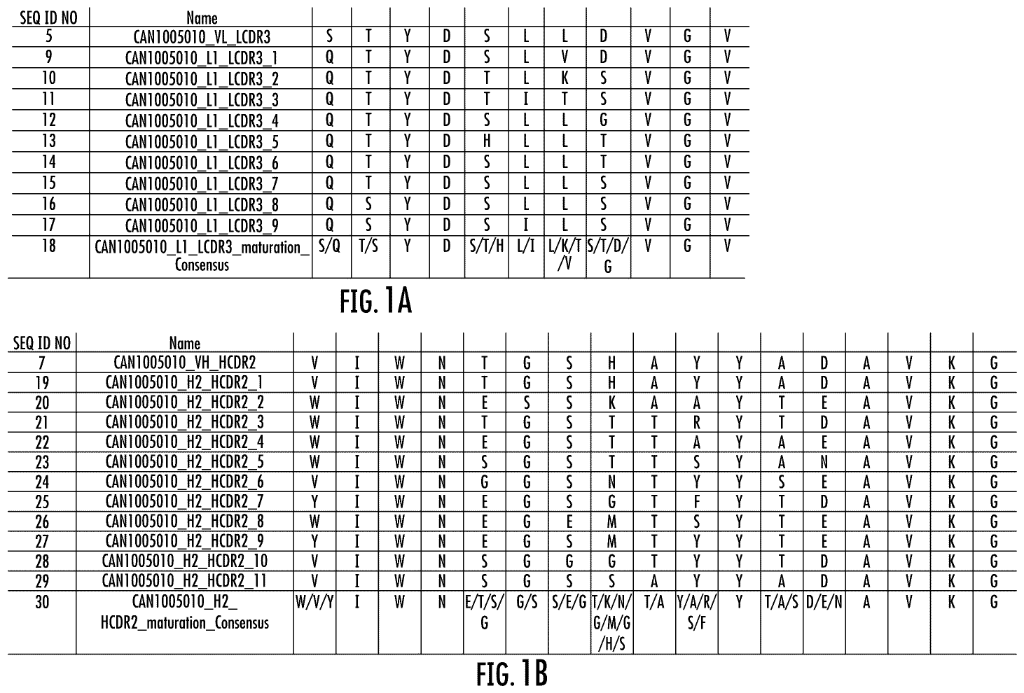

By maturing an antibody having a light chain CAN1005010_VL variable domain according to SEQ ID No.: 1, with a LCDR3 according to SEQ ID No.: 5 (CAN1005010_VL_LCDR3) combined with a heavy chain variable domain according to SEQ ID No.: 2 (CAN1005010_VH) comprising HCDR2 according to SEQ ID No.: 7 (CAN1005010_VH_HCDR2), several high affinity antibodies comprising LCDR3 regions having an amino acid sequence selected from any one of SEQ ID No.: 9, 10, 11, 12, 13, 14, 15, 16, and 17; and a HCDR2 region having an amino acid sequence selected from any one of SEQ ID No.: 19, 20, 21, 22, 23, 34, 25, 26, 27, 28, and 29 were obtained. Based on these LCDR3 regions, a first anti-PD-L1 high affinity LCDR3 consensus sequence according to SEQ ID No.: 18, and a first anti-PD-L1 high affinity HCDR2 consensus sequence according to SEQ ID No.: 30 could be identified by the inventors. Alignments leading to LCDR3 consensus sequence according to SEQ ID No.: 18 and HCDR2 consensus sequence according to SEQ ID No.: 30 are shown in FIG. 1.

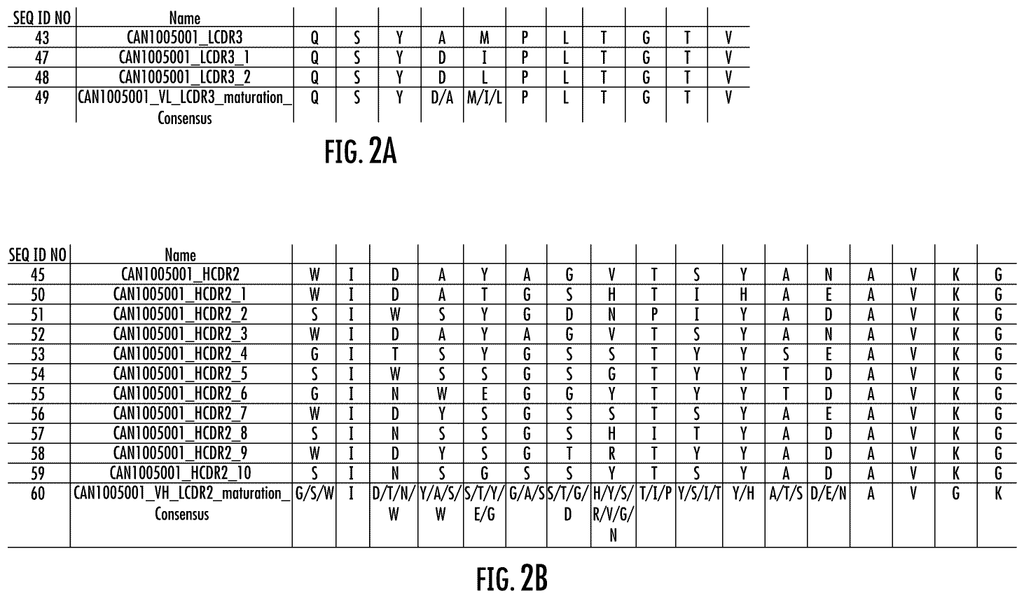

In an alternative aspect of the invention, by maturing an antibody having a light chain variable domain according to SEQ ID No.: 41 (CAN1005001_VL), comprising LCDR3 according to SEQ ID No.: 43 (CAN1005001_VL_LCDR3) combined with a heavy chain variable domain according to SEQ ID No.: 42 (CAN1005001_VH) comprising HCDR2 according to SEQ ID No.: 45 (CAN1005001_VH_HCDR2), several high affinity antibodies comprising LCDR3 regions having an amino acid sequence selected from any one of SEQ ID No.: 47, and 48, and a HCDR2 region having an amino acid sequence selected from any one of SEQ ID No.: 50, 51, 52, 53, 54, 55, 56, 57, 9, and 59, preferably SEQ ID No.: 19, or 52 were obtained. Based on these LCDR3 and HCDR3 regions, a second anti-PD-L1 high affinity LCDR3 consensus sequence according to SEQ ID No.: 49, and a second anti-PD-L1 high affinity HCDR2 consensus sequence according to SEQ ID No.: 60 could be identified by the inventors. Alignments leading to LCDR3 consensus sequence according to SEQ ID No.: 49 and HCDR2 consensus sequence according to SEQ ID No.: 60 are shown in FIG. 2.

According to one aspect, antibody, or antibody fragment according to the present invention may comprise a LCDR3 region having an amino acid sequence selected from any one of SEQ ID No.: 5, 9, 10, 11, 12, 13, 14, 15, 16, 17, 43, 47, and 48. Preferably the antibody comprises an LCDR3 according to SEQ ID No.: 9 or SEQ ID No.: 47.

Furthermore, the antibody or antibody fragment according the present invention may comprise a HCDR2 region having an amino acid sequence selected from any one of SEQ ID No.: 7, 19, 20, 21, 22, 23, 24, 25, 26, 27, 28, 29, 50, 51, 52, 53, 54, 55, 56, 57, 58, and 59. Preferably the antibody comprises an HCDR2 according to SEQ ID No.: 19 or 52.

In one embodiment the variable light chain may comprise a LCDR1 according to SEQ ID No.: 3, a LCDR2 region according to SEQ ID No.: 4, and a LCDR3 region selected from any one of SEQ ID No.: 5, 9, 10, 11, 12, 13, 14, 15, 16, 17, preferably SEQ ID No.: 5. This light chain may for example be combined with another independent embodiment of a variable heavy chain HCDR1 region according to SEQ ID No.: 6, HCDR2 region having an amino acids sequence selected from any one of SEQ ID No.: 7, 19, 20, 21, 22, 23, 24, 25, 26, 27, 28, 29, preferably SEQ ID No.: 19, and a HCDR3 region according to SEQ ID No.: 8.

Accordingly, a highly preferred embodiment of the invention relates to an antibody or antibody fragment comprising a variable light chain comprising a LCDR1 according to SEQ ID No.: 3, a LCDR2 region according to SEQ ID No.: 4, and a LCDR3 region according to SEQ ID No.: 5 combined with a variable heavy chain HCDR1 region according to SEQ ID No.: 6, HCDR2 region according to SEQ ID No.: 19, and a HCDR3 region according to SEQ ID No.: 8.

In one embodiment the light chain may comprise a LCDR1 region according to SEQ ID No.: 3, a LCDR2 region according to SEQ ID No.: 4, and a LCDR3 region according to SEQ ID No.: 43, 47, 48, preferably SEQ ID No.: 47. This light chain may for example be combined with the embodiment of a variable heavy chain HCDR1 region according to SEQ ID No.: 44, HCDR2 region having an amino acids sequence selected from any one of SEQ ID No.: 45, 50, 51, 52, 53, 54, 55, 56, 57, 9, 59, preferably SEQ ID No.: 52 and HCDR3 region according to SEQ ID No.: 46.

Accordingly, a highly preferred embodiment of the invention relates to an antibody or antibody fragment comprising a light chain comprising a LCDR1 region according to SEQ ID No.: 3, a LCDR2 region according to SEQ ID No.: 4, and a LCDR3 region according to SEQ ID No.: 47 combined with a variable heavy chain HCDR1 region according to SEQ ID No.: 44, HCDR2 region according to SEQ ID No.: 52 and HCDR3 region according to SEQ ID No.: 46.

In a preferred embodiment the antibodies or antibody fragments comprise a variable light chain comprising a LCDR3 region according to SEQ ID No.: 18 combined with a variable heavy chain comprising a HCDR2 region according to SEQ ID No.: 30 or comprising a variable light chain comprising a LCDR3 region according to SEQ ID No.: 49 combined with a variable heavy chain comprising a HCDR2 region according to SEQ ID No.: 60.

More preferably the antibodies or antibody fragments comprise a variable light chain comprising a LCDR1 region according to SEQ ID No.: 3, LCDR2 region according to SEQ ID No.: 4, and a LCDR3 region according to SEQ ID No.: 9, and/or a variable heavy chain comprising a HCDR1 region according to SEQ ID No.: 6, a HCDR2 region according to SEQ ID No.: 19, and a HCDR3 region according to SEQ ID No.: 8; as for example comprised in the CAN1005016_VL (SEQ ID No.: 39) and CAN1005016_VH (SEQ ID No.: 40) variable chains.

In an alternative preferred embodiment the antibodies or antibody fragments comprise a variable light chain comprising a LCDR1 region according to SEQ ID No.: 3, LCDR2 region according to SEQ ID No.: 4, and a LCDR3 region according to SEQ ID No.: 47, and/or a variable heavy chain comprising a HCDR1 region according to SEQ ID No.: 44, a HCDR2 region according to SEQ ID No.: 52, and a HCDR3 region according to SEQ ID No.: 46; as for example comprised in the CAN1005019_VL (SEQ ID No.: 65) and CAN1005019_VH (SEQ ID No.: 66) variable chains.

TABLE 1
Sequences of antibodies and antibody fragments

| SEQ | | |
|---|---|---|
| ID | | |
| NO. | Name | Amino Acid Sequence |
| 1 | CAN1005010_VL | SYVLTQLPSVSVTLRQTARITCGGDSIGSKNVYWYQQKLGQAPVLIIY |
| | | DDSSRPSGIPERFSGANSGNTATLTISGALAEDEADYYCSTYDSLLDVG |
| | | VFGGGTQLTVL |
| 2 | CAN1005010_VH | EVQLVESGGDLVKPGGTLRLSCVASGFTFSSYDMSWVRQSPGKGLQ |
| | | WVAVIWNEGSSTYYADAVKGRFTISRDNAKNTLYLQMNSLRAEDTA |
| | | VYYCARGRQPRLVGTLDLWGQGTLVTVSS |
| 3 | CAN1005010_VL_LCDR1 | GGDSIGSKNVY |
| 4 | CAN1005010_VL_LCDR2 | DDSSRPS |
| 5 | CAN1005010_VL_LCDR3 | STYDSLLDVGV |
| 6 | CAN1005010_VH_HCDR1 | SYDMS |
| 7 | CAN1005010_VH_HCDR2 | VIWNEGSSTYYADAVKG |
| 8 | CAN1005010_VH_HCDR3 | GRQPRLVGTLDL |
| 9 | CAN1005010_L1_LCDR3_1 | QTYDSLVDVGV |
| 10 | CAN1005010_L1_LCDR3_2 | QTYDTLKSVGV |
| 11 | CAN1005010_L1_LCDR3_3 | QTYDTITSVGV |
| 12 | CAN1005010_L1_LCDR3_4 | QTYDSLLGVGV |
| 13 | CAN1005010_L1_LCDR3_5 | QTYDHLLTVGV |
| 14 | CAN1005010_L1_LCDR3_6 | QTYDSLLTVGV |
| 15 | CAN1005010_L1_LCDR3_7 | QTYDSLLSVGV |
| 16 | CAN1005010_L1_LCDR3_8 | QSYDSLLSVGV |
| 17 | CAN1005010_L1_LCDR3_9 | QSYDSILSVGV |
| 18 | CAN1005010_VL_LCDR3_ | (S, Q) (T/S) Y D (S/T/H) (L/I) (L/K/T/V) |
| | maturation_Consensus | (S/T/D/G) V G V |
| 19 | CAN1005010_H2_HCDR2_1 | VIWNTGSHAYYADAVKG |
| 20 | CAN1005010_H2_HCDR2_2 | WIWNESSKAAYTEAVKG |
| 21 | CAN1005010_H2_HCDR2_3 | WIWNTGSTTRYTDAVKG |
| 22 | CAN1005010_H2_HCDR2_4 | WIWNEGSTTAYAEAVKG |
| 23 | CAN1005010_H2_HCDR2_5 | WIWNSGSTTSYANAVKG |
| 24 | CAN1005010_H2_HCDR2_6 | VIWNGGSNTYYSEAVKG |
| 25 | CAN1005010_H2_HCDR2_7 | YIWNEGSGTFYTDAVKG |
| 26 | CAN1005010_H2_HCDR2_8 | WIWNEGEMTSYTEAVKG |
| 27 | CAN1005010_H2_HCDR2_9 | YIWNEGSMTYYTEAVKG |
| 28 | CAN1005010_H2_HCDR2_10 | VIWNSGGGTYYTDAVKG |
| 29 | CAN1005010_H2_HCDR2_11 | VIWNSGSSAYYADAVKG |
| 30 | CAN1005010_VH_ | (W/V/Y) I W N (E/T/S/G) (G/S) (S/E/G) |
| | HCDR2_maturation_ | (T/K/N/G/M/G/H/S) (T/A) (Y/A/R/S/F) Y |
| | Consensus | (T/A/S) (D/E/N) A V K G |
| 31 | CAN1005010_LFW1 | SYVLTQLPSVSVTLRQTARITC |
| 32 | CAN1005010_LFW2 | WYQQKLGQAPVLIIY |
| 33 | CAN1005010_LFW3 | GIPERFSGANSGNTATLTISGALAEDEADYYC |
| 34 | CAN1005010_LFW4 | FGGGTQLTVL |
| 35 | CAN1005010_HFW1 | EVQLVESGGDLVKPGGTLRLSCVAS |
| 36 | CAN1005010_HFW2 | WVRQSPGKGLQWVA |
| 37 | CAN1005010_HFW3 | RFTISRDNAKNTLYLQMNSLRAEDTAVYYCAR |
| 38 | CAN1005010_HFW4 | WGQGTLVTVSS |
| 39 | CAN1005010_L1 or | SYVLTQLPSVSVTLRQTARITCGGDSIGSKNVYWYQQKLGQAPVLIIY |
| | CAN1005016_VL | DDSSRPSGIPERFSGANSGNTATLTISGALAEDEADYYCQTYDSLVDV |
| | | GVFGGGTQLTVL |
| 40 | CAN1005010_H2 or | EVQLVESGGDLVKPGGTLRLSCVASGFTFSSYDMSWVRQSPGKGLQ |
| | CAN1005016_VH | WVAVIWNTGSHAYYADAVKGRFTISRDNAKNTLYLQMNSLRAEDTA |
| | | VYYCARGRQPRLVGTLDLWGQGTLVTVSS |
| 41 | CAN1005001_VL | SYVLTQLPSVSVTLRQTARITCGGDSIGSKNVYWYQQKLGQAPVLIIY |
| | | DDSSRPSGIPERFSGANSGNTATLTISGALAEDEADYYCQSYAMPLTG |
| | | TVFGGGTQLTVL |
| 42 | CAN1005001_VH | EVQLVESGGDLVKPAGSLRLSCVASGFTFSSYSMSWVRQAPEKGLQL |
| | | VAGISSGGSSTYYTDAVKGRFTISRDNAKNTVYLQMNSLRAEDTAMY |
| | | YCARGQGGRSSFDYWGQGTLVTVSS |
| 43 | CAN1005001_LCDR3 | QSYAMPLTGTV |
| 44 | CAN1005001_HCDR1 | SYSMS |
| 45 | CAN1005001_HCDR2 | WIDAYAGVTSYANAVKG |
| 46 | CAN1005001_HCDR3 | GQGGRSSFDY |
| 47 | CAN1005001_LCDR3_1 | QSYDIPLTGTV |
| 48 | CAN1005001_LCDR3_2 | QSYDLPLTGTV |
| 49 | CAN1005001_VL_LCDR3_ | QSY(D/A)(M/I/L)PLTGTV |
| | maturation_Consensus | |
| 50 | CAN1005001_HCDR2_1 | WIDATGSHTIHAEAVKG |
| 51 | CAN1005001_HCDR2_2 | SIWSYGDNPIYADAVKG |
| 52 | CAN1005001_HCDR2_3 | WIDAYAGVTSYANAVKG |
| 53 | CAN1005001_HCDR2_4 | GITSYGSSTYYSEAVKG |
| 54 | CAN1005001_HCDR2_5 | SIWSSGSGTYYTDAVKG |
| 55 | CAN1005001_HCDR2_6 | GINWEGGYTYYTDAVKG |
| 56 | CAN1005001_HCDR2_7 | WIDYSGSSTSYAEAVKG |
| 57 | CAN1005001_HCDR2_8 | SINSSGSHITYADAVKG |
| 58 | CAN1005001_HCDR2_9 | WIDYSGTRTYYADAVKG |
| 59 | CAN1005001_HCDR2_10 | SINSGSSYTSYADAVKG |
| 60 | CAN1005001_VH_HCDR2_ | (G/S/W) I (D/T/N/W) (Y/A/S/W) (S/T/Y/E/G) |
| | maturation_Consensus | (G/A/S) (S/T/G/D) (H/Y/S/R/V/G/N) (T/I/P) |
| | | (Y/S/I/T) (Y/H) (A/T/S) (D/E/N) AVGK |
| 61 | CAN1005001_HFW1 | EVQLVESGGDLVKPAGSLRLSCVASGFTFS |
| 62 | CAN1005001_HFW2 | WVRQAPEKGLQLVA |
| 63 | CAN1005001_HFW3 | RFTISRDNAKNTVYLQMNSLRAEDTAMYYCAR |
| 64 | CAN1005001_HFW4 | WGQGTLVTVSS |
| 65 | CAN1005001_L1 or | SYVLTQLPSVSVTLRQTARITCGGDSIGSKNVYWYQQKLGQAPVLIIY |
| | CAN1005019_VL | DDSSRPSGIPERFSGANSGNTATLTISGALAEDEADYYCQSYDIPLTGT |
| | | VFGGGTQLTVL |
| 66 | CAN1005001_H3 or | EVQLVESGGDLVKPAGSLRLSCVASGFTFSSYSMSWVRQAPEKGLQL |
| | CAN1005019_VH | VAWIDAYAGVTSYANAVKGRFTISRDNAKNTVYLQMNSLRAEDTAM |
| | | YYCARGQGGRSSFDYWGQGTLVTVSS |
| 67 | PD-L1 | FTITVSKDLYVVEYGGNVTMECKFPVEKQLNLFALIVYWEMEDKKIIQ |
| | | FVNGKEDLKVQHSSYSQRAQLLKDQLFLGKAALQITDVRLQDAGVY |
| | | CCLIGYGGADYKRITLKVHAPYRNISQRISVDPVTSEHELMCQAEGYP |
| | | EAEVIWTSSDHRVLSGKTTITNSNREEKLFNVTSTLNINATANEIFYCTF |
| | | QRSGPEENNTAELVIPERLPVPASERTHFMILGPFLLLLGVVLAVTFCL |
| | | KKHGRMMDVEKCCTRDRNSKKRNDIQFEET |

In consensus sequences 18, 30, 49 and 60, the sequences may comprise any of amino acids for a specific position in a bracket and separated by/“as alternatives.” For example, position (G/S/W) in SEQ ID No 60 may be a glycine, a serine, or a tryptophane.

Further to the CDR1, CDR2, and CDR3 regions, the variable light and heavy domains comprise framework regions 1 to 4 from N-terminal to C-terminal direction. The antibodies or antibody fragments according to the invention may comprise one, several, or all of the light chain variable domain framework sequences having an amino acid sequence selected from any one of SEQ ID No.: 31, 32, 33, 34; and/or one, several, or all of the heavy chain variable domain framework sequences having an amino acid sequence selected from any one of SEQ ID No.: 35, 36, 37, 38 or one, several, or all of the heavy chain variable domain framework sequences having an amino acid sequence selected from any one of SEQ ID No.: 61, 62, 63, 64. A heavy chain variable domain comprising HCDR1 and HCDR3 according to SEQ ID Nos 6 and 8, and a HCDR2 sequence selected from any one of SEQ ID No.: 5, 9, 10, 11, 12, 13, 14, 15, 16, 17 preferably comprises framework regions selected from any one of SEQ ID No.: 35, 36, 37, 38, and a heavy chain variable domain comprising HCDR1 and HCDR3 according to SEQ ID Nos 44 and 46, and a HCDR2 sequence selected from any one of SEQ ID No.: 51, 51, 52, 53, 54, 55, 56, 57, 58, 59, preferably comprises framework regions selected from any one of SEQ ID No.: 61, 62, 63, and 64.

In a preferred embodiment, the antibody or antibody fragment according to any of the present invention comprises a light chain variable domain according to SEQ ID No.: 1, or 41, more preferably SEQ ID No.: 39 or 65, and/or a heavy chain variable domain according to SEQ ID No.: 2, or 42, preferably SEQ ID No.: 40, or 66, more preferably a light chain variable domain according to SEQ ID No.: 39 combined with a heavy chain variable domain according to SEQ ID No.: 40, or a light chain variable domain according to SEQ ID No.: 65 combined with a heavy chain variable domain according to SEQ ID No.: 66.

The invention further provides antibodies or antibody fragments as described herein, wherein the antibody or antibody fragment comprises at least one LCDR, HCDR, framework region, or variable domain chain having a sequence having at least 85%, at least 90%, at least 95%, at least 97%, at least 98%, or at least 99% sequence identity to the amino acid sequences of SEQ ID No.: 1 to SEQ ID No.: 66. Such antibodies or antibody fragments may be provided by introducing at least one, for example one, two, three, our four mutations in form of an amino acid substitution or amino acid deletion into the amino acid sequences of SEQ ID No.: 1 to SEQ ID No.: 66. Preferably, the mutation is a conservative amino acid substitution.

In a further aspect the invention relates to an antibody or antibody fragment which cross-competes with an antibody fragment described above, preferably with an antibody or antibody fragment comprising a light chain variable domain according to SEQ ID No.: 39, and/or a heavy chain variable domain according to SEQ ID No.: 40; or comprising a light chain variable domain according to SEQ ID No.: 65, and/or a heavy chain variable domain according to SEQ ID No.: 66.

The antibody or antibody fragment according to any of the preceding claims, wherein the antibody or antibody fragment specifically binds to canine PD-L1 with a dissociation constant (Kd) of less than about 20 nM, 18 nM, 16 nM, 14 nM, 12 nM, preferably less than about 10 nM, 8 nM, 6 nM, more preferably less than about 5 nM, 4 nM, 5 nM, most preferably less than about 2 nM. Dissociation constant is determined for the binding of the IgG antibody to the monovalent PD-L1 antigen. The canine PD-L1 to which the disclosed antibodies and antibody fragments bind for example by the is characterised amino acid sequence of FTITVSKDLYVVEYGGNVTMECKFPVEKQLNLFALIVYWEMEDKKIIQFVNGKEDLKVQHSSYSQRAQ LLKDQLFLGKAALQITDVRLQDAGVYCCLIGYGGADYKRITLKVHAPYRNISQRISVDPVTSEHELMCQ AEGYPEAEVIWTSSDHRVLSGKTTITNSNREEKLFNVTSTLNINATANEIFYCTFQRSGPEENNTAEL VIP ERLPVPASERTHFMILGPFLLLLGVVLAVTFCLKKHGRMMDVEKCCTRDRNSKKRNDIQFEET (SEQ ID No.: 67) in accordance with NCBI Reference Sequence: NP_001278901.1.

The antibody may bind to canine PD-L1 with an Kon rate of at least about 2× 10⁴ [M⁻¹s⁻¹], preferably least about 5× 10+ [M⁻¹s⁻¹], more preferably least about 1×10⁵ [M⁻¹s⁻¹]; and/or with a Koff rate lower that about 1× 10⁻³ [s⁻¹], preferably lower that about 5× 10-+ [s⁻¹], or lower that about 2.5× 10-+ [s⁻¹]. In one embodiment the antibody has a Kon of at least about 1× 10⁵ [M⁻¹s⁻¹] and a Koff lower that about 2.5× 10-+ [s⁻¹]. Dissociation constant is determined for the binding of the IgG antibody to the monovalent PD-L1 antigen. In another embodiment the antibody has a Kon of at least about 7.5× 10+ [M-Is⁻¹] and a Koff lower that about 2.5× 10+ [s⁻¹]. Dissociation constant is determined for the binding of the IgG antibody to the monovalent PD-L1 antigen.

The antibody or antibody fragment according to the present invention may interfere or block the interaction between canine PD-L1 (cPD-L1) and canine PD-1 (cPD-1) as confirmed by Example 8. For example, the antibody or antibody fragment may block the interaction between cPD-L1 and cPD-1 with an IC50 value of lower than about 100 nM, preferably lower than about 50 nM, or lower than 20 about nM, more preferably lower than about 10, or lower than about 5 nM, most preferably lower than about 3 nM, or even lower than about 2 nM. IC50 values for the cPD-L1 and cPD-1 interaction may for example be determined by biolayer-Interferometry (BLI)-based ligand-binding inhibition assay or ELISA ligand-binding inhibition assay (ELISA Ibia).

According to a further aspect, the antibody or antibody fragment according to the invention is an antibody fragment selected from Fv, scFv, Fab, Fab′, F(ab′) 2, Fab′-SH, or VHH.

The antibody or antibody fragment according to any of the present invention may comprise an Fc fragment or fragment thereof selected from canine isotype A of immunoglobulin G (also termed HC-A, HCA, calgG-A), isotype B of immunoglobulin G (also termed HC-B, HCB, calgG-B), isotype C of immunoglobulin G (also termed HC-C, HCC, calgG-C), or isotype D of immunoglobulin G (also termed HC-D, HCD, calgG-D). Preferably the Fc domain is selected from isotype B. The Fc domain comprised in the antibody or antibody fragment may have a wild-type sequence or a mutated sequence.

Depending on the therapeutic application, the selection of the respective antibody isotype is crucial, and it needs to consider whether engagement of humoral or cellular components of the immune system is advantageous or even might lead to unwanted side effects of a drug. For example, a therapeutic antibody against tumor cell growth or a pathogen should have strong effector functions. In contrast, targeting soluble mediators or cell surface receptors of a healthy cell to prevent receptor-ligand interactions typically requires absence of any CDC or ADCC activity to prevent target cell death or unwanted cytokine secretion. Disease areas in which silent antibody formats are necessary contain but are not limited to inflammatory diseases (e.g., rheumatoid arthritis, psoriasis, inflammatory bowel disease), allergies (e.g., asthma), pain (e.g., osteoarthritic pain, cancer pain, lower back pain) and eye disease (e.g., age related macular degeneration). Depending on the target, the absence of CDC or ADCC may also be desirable in a antibody for the treatment of cancer, such as an antibody targeting PD-L1 in accordance with the present invention.

The term “Fc fragment” relates to a fragment of an immunoglobulin comprising at least parts of, or the entire constant heavy chain region 2 (C2 or CH2) and constant heavy chain region 3 (C3 or CH3) or of the crystallisable fragment of an immunoglobulin obtained by papain digestion. The fragment is understood to be a part of a larger polypeptide sequence. Thus the “fragment” will usually have amino acids sequence bound to the C-and/or N-terminus. The terms “C2” or “CH2” as well as terms “C3” or “CH3” may be used interchangeably. Furthermore, the terms “Fc region” and “Fc domain” may be used interchangeably when referring to the immunoglobulin Fc CH2 and CH3 sequences unless explicitly stated otherwise. Within the context of the present invention, the boundaries of the CH2 and CH3 region for canine immunoglobulin isotypes HC-A, HC—B, HC—C and HC-D are defined according to Tang et al. (Tang L, Sampson C, Dreitz MJ, McCall C (2001) Cloning and characterization of cDNAs encoding four different canine immunoglobulin gamma chains. Vet Immunol Immunopathol. 80 (3-4): 259-70), which is incorporated herein by reference.

The wild-type Fc fragment of the different isotypes may have a sequence as disclosed in Table 2.

TABLE 2
Wild-type canine Fc sequences of different isotypes

| | SEQ | |
|---|---|---|
| canine | ID | |
| isotype | NO: | Sequence |
| HC-A | 68 | ASTTAPSVFPLAPSCGSTSGSTVALACLVSGYFPEP |
| | | VTVSWNSGSLTSGVHTFPSVLQSSGLHSLSSMVTVP |
| | | SSRWPSETFTCNVVHPASNTKVDKPVFNECRCTDTP |
| | | PCPVPEPLGGPSVLIFPPKPKDILRITRTPEVTCVV |
| | | LDLGREDPEVQISWFVDGKEVHTAKTQSREQQFNGT |
| | | YRVVSVLPIEHQDWLTGKEFKCRVNHIDLPSPIERT |
| | | ISKARGRAHKPSVYVLPPSPKELSSSDTVSITCLIK |
| | | DFYPPDIDVEWQSNGQQEPERKHRMTPPQLDEDGSY |
| | | FLYSKLSVDKSRWQQGDPFTCAVMHETLQNHYTDLS |
| | | LSHSPGK |
| | | GenBank accession No. AF354264 |
| | | WO2012153126 A1 ID NO: 8 |
| HC-B | 69 | ASTTAPSVFPLAPSCGSTSGSTVALACLVSGYFPEP |
| | | VTVSWNSGSLTSGVHTFPSVLQSSGLYSLSSMVTVP |
| | | SSRWPSETFTCNVAHPASKTKVDKPVPKRENGRVPR |
| | | PPDCPKCPAPEMLGGPSVFIFPPKPKDTLLIARTPE |
| | | VTCVVVDLDPEDPEVQISWFVDGKQMQTAKTQPREE |
| | | QFNGTYRVVSVLPIGHQDWLKGKQFTCKVNNKALPS |
| | | PIERTISKARGQAHQPSVYVLPPSREELSKNTVSLT |
| | | CLIKDFFPPDIDVEWQSNGQQEPESKYRTTPPQLDE |
| | | DGSYFLYSKLSVDKSRWQRGDTFICAVMHEALHNHY |
| | | TQESLSHSPGK |
| | | GenBank accession No. AF354265 |
| | | WO 2010/027488 A2, SEQ ID NO: 54 |
| | | WO 2012/153126 A1, SEQ ID NO: 9 |
| HC-C | 70 | ASTTAPSVFPLAPSCGSQSGSTVALACLVSGYIPEP |
| | | VTVSWNSVSLTSGVHTFPSVLQSSGLYSLSSMVTVP |
| | | SSRWPSETFTCNVAHPATNTKVDKPVAKECECKCNC |
| | | NNCPCPGCGLLGGPSVFIFPPKPKDILVTARTPTVT |
| | | CVVVDLDPENPEVQISWFVDSKQVQTANTQPREEQS |
| | | NGTYRVVSVLPIGHQDWLSGKQFKCKVNNKALPSPI |
| | | EEIISKTPGQAHQPNVYVLPPSRDEMSKNTVTLTCL |
| | | VKDFFPPEIDVEWQSNGQQEPESKYRMTPPQLDEDG |
| | | SYFLYSKLSVDKSRWQRGDTFICAVMHEALHNHYTQ |
| | | ISLSHSPGK |
| | | GenBank accession No. AF354266 |
| | | WO 2012/153126 A1, SEQ ID NO: 10 |
| HC-D | 71 | ASTTAPSVFPLAPSCGSTSGSTVALACLVSGYFPEP |
| | | VTVSWNSGSLTSGVHTFPSVLQSSGLYSLSSTVTVP |
| | | SSRWPSETFTCNVVHPASNTKVDKPVPKESTCKCIS |
| | | PCPVPESLGGPSVFIFPPKPKDILRITRTPEITCVV |
| | | LDLGREDPEVQISWFVDGKEVHTAKTQPREQQFNST |
| | | YRVVSVLPIEHQDWLTGKEFKCRVNHIGLPSPIERT |
| | | ISKARGQAHQPSVYVLPPSPKELSSSDTVTLTCLIK |
| | | DFYPPEIDVEWQSNGQPEPESKYHTTAPQLDEDGSY |
| | | FLYSKLSVDKSRWQQGDTFTCAVMHEALQNHYTDLS |
| | | LSHSPGK |
| | | WO 2012/153126 A1, SEQ ID NO: 11 |

The Fc fragment comprised in the antibody or antibody fragment may further comprise a substitution in any of the wild-type sequences disclosed in Table 2. Substitutions that may be comprised are disclosed in WO 2021/165417A1 which is incorporated herein by reference. Specifically, the Fc fragment comprises at least one substitution of an amino acid selected from at least one of amino acid positions 235, 239, 270, and/or 331 relative to the wild-type Fc fragment. Preferably the mutation is in an Fc fragment from isotype B of canine IgG.

Preferably, the Fc fragment, comprises least two substitutions of amino acids selected from at least two of the amino acids at positions 234, 235, 239, 270, and/or 331. More preferably, the two amino acids are 235 and 239; 235 and 270; 235 and 331; 239 and 270; 239 and 331; 270 and 331, 234 and 235, 234 and 239; 234 and 270; or 234 and 331.

In another preferred embodiment the Fc fragment, wherein the Fc fragment comprises least three substitutions of amino acids selected from at least three of amino acid positions 234, 235, 239, 270, and/or 331. More preferably, the three amino acid positions are 235, 239, and 270; 239, 270, and 331; 235, 270, and 331; or 235, 239, and 331 in a wild-type Fc sequence disclosed in Table 2.

In another preferred embodiment the present invention relates to a polypeptide comprising at least a canine or feline Fc fragment, wherein the Fc fragment comprises at least four substitutions of amino acids selected from amino acid positions 234, 235, 239, 270, and 331, more preferably 235, 239, 270, and 331, and most preferably amino acids L235, S239, D270, and P331. Most preferably the Fc fragment comprises the mutations L235A, S239A, D270A, and P331G relative to the wild-type Fc fragment.

The respective substitutions in the Fc Fragment led to a reduced binding affinity to Clq and/or an Fc receptor relative to a polypeptide comprising the corresponding wild-type Fc fragment. Under physiological conditions of an uncompromised immune system, reduced binding, or diminished binding to Clq and/or FcγRI results in a reduction or complete elimination of the immune effector functions of the complement-dependent cytotoxicity (CDC) and induction of antibody-dependent cytotoxicity (ADCC). The reduced binding or diminished binding of polypeptides comprising at least one substitution in the Fc fragment to Clq and/or FcγRI and/or the resulting reduction or complete elimination of CDC or ADCC is also referred commonly referred to as silencing. In the Fc fragment of a canine isotype B, the Fc fragment maintains its ability to bind to neonatal Fc receptor (FcRn) as well as to Protein A.

The antibody or antibody fragment may comprise the above Fc fragments with reduced or completely eliminated CDC or ADCC preferably for use in inflammatory diseases, allergies, pain, and eye disease.

In a preferred embodiment, the antibody comprises a light chain comprising light chain variable domain (VL) as disclosed herein and a lambda constant domain (CL) and a heavy chain comprising heavy chain variable domain (VH) connected to the Fc fragment (comprising a CH-2 and a CH-3 domain) as described above by a CH-domain. The CH-1 domain may be connected to the CH-2 domain via an amino acid sequence referred to as the “hinge” or alternatively as the “hinge region”. Suitable canine lambda constant domain (CL) and canine CH-1 domains are known in the art. The two heavy chains are preferably linked to each other by disulfide bonds and each heavy chain is preferably linked to one of the light chains also through a disulfide bond.

In specific embodiments of the invention the antibodies comprising a light chain and a heavy chain may comprise a light chain according to SEQ ID No: 72, or 74 and (or a heavy chain according to SEQ ID No: 73, or 75, preferably a light chain according to SEQ ID No: 72 combined with a heavy chain according to SEQ ID No: 73, or a light chain according to SEQ ID No: 74 combined with a heavy chain according to SEQ ID No: 75.

TABLE 3
Full length antibody sequences:

| SEQ ID | | |
|---|---|---|
| NO. | Name | Sequence |
| 72 | pCaMx_IgG1_ | MVLQTQVFISLLLWISGAYASYVLTQLPSVS |
| | lambda_3B_ | VTLRQTARITCGGDSIGSKNVYWYQQKLGQA |
| | CAN1005016_ | PVLIIYDDSSRPSGIPERFSGANSGNTATLT |
| | silent_ | ISGALAEDEADYYCQTYDSLVDVGVFGGGTQ |
| | light | LTVLGQPKASPSVTLFPPSSEELGANKATLV |
| | | CLISDFYPSGVTVAWKADGSPVTQGVETTKP |
| | | SKQSNNKYAASSYLSLTPDKWKSHSSFSCLV |
| | | THEGSTVEKKVAPAECS |
| 73 | pCaMx_IgG1_ | MKHLWFFLLLVAAPRWVLSEVQLVESGGDLV |
| | lambda_3B_ | KPGGTLRLSCVASGFTFSSYDMSWVRQSPGK |
| | CAN1005016_ | GLQWVAVIWNTGSHAYYADAVKGRFTISRDN |
| | silent_ | AKNTLYLQMNSLRAEDTAVYYCARGRQPRLV |
| | heavy | GTLDLWGQGTLVTVSSASTTAPSVFPLAPSC |
| | | GSTSGSTVALACLVSGYFPEPVTVSWNSGSL |
| | | TSGVHTFPSVLQSSGLYSLSSMVTVPSSRWP |
| | | SETFTCNVAHPASKTKVDKPVPKRENGRVPR |
| | | PPDCPKCPAPEMAGGPSVFIFPPKPKDTLLI |
| | | ARTPEVTCVVVDLDPEDPEVQISWFVDGKQM |
| | | QTAKTQPREEQFNGTYRVVSVLPIGHQDWLK |
| | | GKQFTCKVNNKALPSGIERTISKARGQAHQP |
| | | SVYVLPPSREELSKNTVSLTCLIKDFFPPDI |
| | | DVEWQSNGQQEPESKYRTTPPQLDEDGSYFL |
| | | YSKLSVDKSRWQRGDTFICAVMHEALHNHYT |
| | | QESLSHSPGK |
| 74 | pCaMx_IgG1_ | MVLQTQVFISLLLWISGAYASYVLTQLPSVS |
| | lambda_3A_ | VTLRQTARITCGGDSIGSKNVYWYQQKLGQA |
| | CAN1005019_ | PVLIIYDDSSRPSGIPERFSGANSGNTATLT |
| | silent_ | ISGALAEDEADYYCQSYDIPLTGTVFGGGTQ |
| | light | LTVLGQPKASPSVTLFPPSSEELGANKATLV |
| | | CLISDFYPSGVTVAWKADGSPVTQGVETTKP |
| | | SKQSNNKYAASSYLSLTPDKWKSHSSFSCLV |
| | | THEGSTVEKKVAPAECS |
| 75 | pCaMx_IgG1_ | MKHLWFFLLLVAAPRWVLSEVQLVESGGDLV |
| | lambda_3A_ | KPAGSLRLSCVASGFTFSSYSMSWVRQAPEK |
| | CAN1005019_ | GLQLVAWIDAYAGVTSYANAVKGRFTISRDN |
| | silent_ | AKNTVYLQMNSLRAEDTAMYYCARGQGGRSS |
| | heavy | FDYWGQGTLVTVSSASTTAPSVFPLAPSCGS |
| | | TSGSTVALACLVSGYFPEPVTVSWNSGSLTS |
| | | GVHTFPSVLQSSGLYSLSSMVTVPSSRWPSE |
| | | TFTCNVAHPASKTKVDKPVPKRENGRVPRPP |
| | | DCPKCPAPEMAGGPSVFIFPPKPKDTLLIAR |
| | | TPEVTCVVVDLDPEDPEVQISWFVDGKQMQT |
| | | AKTQPREEQFNGTYRVVSVLPIGHQDWLKGK |
| | | QFTCKVNNKALPSGIERTISKARGQAHQPSV |
| | | YVLPPSREELSKNTVSLTCLIKDFFPPDIDV |
| | | EWQSNGQQEPESKYRTTPPQLDEDGSYFLYS |
| | | KLSVDKSRWQRGDTFICAVMHEALHNHYTQE |
| | | SLSHSPGK |

In a preferred aspect of the invention antibody or antibody fragment is a fully canine antibody or antibody fragment. In a preferred aspect of the invention antibody or antibody fragment is an isolated antibody or antibody fragment. Preferably antibody or antibody fragment the according to the invention is a monoclonal antibody or antibody fragment.

In a further aspect the antibody or antibody fragment is a recombinant antibody or antibody fragment. A “recombinant” antibody is an antibody that is produced in cells of a different species than the species from which genome the antibody is derived. Suitable cells for recombinantly expressing an antibody according to the invention especially include mammalian cells, such as primate or non-primate animal cells; yeast cells; plant cells; and insect cells. Non-limiting exemplary mammalian cells include, but are not limited to, NSO cells, 293 cells, and CHO cells, and cell lines derived therefrom, for example 293-6E, DG44, CHO-S, and CHO-K cells and hybridoma cells. Within the context of the present invention, also a synthetic or semisynthetic derived antibody is considered a recombinant antibody.

In a further aspect, the invention relates to a pharmaceutical composition comprising the antibody or antibody fragment as described herein, optionally together with a pharmaceutical acceptable carrier or excipient. A “pharmaceutical composition” is a composition comprising the antibody or antibody fragment according to the invention and additional compounds which are toxicologically acceptable and enable the storage and the administration of the antibody or antibody fragment according to the invention to a subject to be treated and allows the antibody or antibody fragment to exert its intended pharmacological and biological activity.

The pharmaceutically acceptable carrier or excipient may include agents, e.g., diluents, stabilizers, adjuvants, or other types of excipients that are non-toxic to the cell or mammal to be exposed thereto at the dosages and concentrations employed. Examples of pharmaceutically acceptable carriers include alumina; aluminum stearate; lecithin; serum proteins, such as human serum albumin, canine or other animal albumin; buffers such as phosphate, citrate, tromethamine or HEPES buffers; glycine; sorbic acid; potassium sorbate; partial glyceride mixtures of saturated vegetable fatty acids; water; salts or electrolytes, such as protamine sulfate, disodium hydrogen phosphate, potassium hydrogen phosphate, sodium chloride, zinc salts, colloidal silica, or magnesium trisilicate; polyvinyl pyrrolidone, cellulose-based substances; polyethylene glycol; sucrose; mannitol; or amino acids including, but not limited to, arginine.

The invention further relates to the antibody or antibody fragment, or the pharmaceutical compositions described herein for use in the treatment or method of treatment of a subject, preferably a canine subject, in need thereof. Preferably the treatment or method of treatment is for treating a disease. A method of treating a disease according to the invention encompasses a step of administering the antibody or antibody fragment or the pharmaceutical compositions described herein to a patient in need of treatment, preferably to a canine subject. Preferably the disease is cancer or an inflammatory or autoimmune disease. Preferably the cancer is colorectal cancer, Melanoma, oral malignant melanoma, osteosarcoma, mastocytoma, angiosarcoma, hepatocellular sarcoma, squamous carcinoma, nasal adenocarcinoma, transitional cell carcinoma, anal sac gland carcinoma, soft tissue carcinoma, mammary adenocarcinoma, histiocytic sarcoma, diffuse large B-cell carcinoma, gastric cancer, for example gastric adenocarcinoma, lymphoma, non-small cell lung cancer, small cell lung cancer, renal cell carcinoma, head and neck cancer.

According to a further aspect, the antibody or antibody fragment described herein may be used in a diagnostic assay, diagnostic method, and the like. The method or assay may include a step of detecting canine PD-L1.

In a further aspect, the invention relates to a polynucleotide or plurality of polynucleotides encoding the antibody or antibody fragment according to the invention. The polynucleotide or plurality of polynucleotides may be an isolated polynucleotide. The polynucleotide or plurality of polynucleotides may be comprised in a vector, such as a plasmid or an artificial chromosome. The polynucleotide may be operatively linked to transcriptional and translational control sequences. In this context, the term “operatively linked” means that a transcriptional and translational control sequences serve to functionally transcribe and translate the antibody or antibody fragment to express the encoded antibody or antibody fragment.

The vector and/or the polynucleotide or plurality of polynucleotides may be comprised in a cell. The cell is preferably a host cell suitable for recombinantly expressing antibodies or antibody fragments. Exemplary eukaryotic cells include mammalian cells, such as primate or non-primate animal cells; yeast cells; plant cells; and insect cells. Non-limiting exemplary mammalian cells include, but are not limited to, NSO cells, 293 cells, and CHO cells, and cell lines derived therefrom, for example 293-6E, DG44, CHO-S, and CHO-K cells.

### EXAMPLES

Generation of canine antibodies specific for canine PD-L1

A collection of canine antibodies that specifically bind canine PD-L1 were generated. The sequences of the respective antibodies were isolated from a synthetic fully canine antibody library disclosed in WO 2018/234438A1 using phage display. The obtained antibodies inhibit the binding of canine PD-1 to canine PD-L1. Affinity maturation was performed on individual inhibitory clones in order to increase binding affinity while maintaining epitope specificity.

### Example 1

Generation and screening of anti-canine PD-L1 antibodies

Phage display selections may be done as described below or by another method known to one of skill in the art. To increase the likelihood of discovering diverse binding antibodies, different panning strategies were applied (e.g., solid phase panning, solution panning, semi-solution panning, Fc-capture panning). Selection against cPD-L1 as an Fc-fusion protein (cPD-L1_hFc; Sino Biological; Cat: 70110-D02H) was performed in 3 rounds in solid phase panning, as well as in solution and semi-solution panning, as described below. Due to the nature of the antigen as an Fc-fusion protein, phages were blocked against human IgG to eliminate potential Fc-reactive candidates. Common to all panning strategies employed is that the antigen amount was reduced with each round, whereas washing stringency was increased. Phage outputs after 3 rounds of panning were subcloned into a bacterial Fab expression vector prior to screening in ELISA format.

### Solid Phase Panning

When performing solid phase panning, cPD-L1_hFc was immobilized on the surface of a microtiter plate (Maxisorp, 96-well flat bottom) for at least 1 hour at RT. cPD-L1 was used at a concentration of 3 μg/ml in the first round of panning, together with 5 μg/ml human serum IgG (hulgG; Jackson ImmunoResearch; Cat.: 009-000-003) for blocking, which was also immobilized on the plate in parallel. For each selection, 250 μl/well of antigen diluted in PBS (Phosphate buffered saline, pH 7.4) were used. After incubation, the wells were washed with PBST (PBS supplemented with 0.05% Tween-20) before addition of 300 μl/well blocking buffer (Chemiblocker, Merck-Millipore).

In parallel, library phages were blocked for 1 h at RT with blocking buffer. Due to the hFc-tag, 10 μg/ml human serum IgG was added to the blocking buffer.

For each panning subcode, 20 ml 2× YT medium were incubated with E. coli ER2738 from a M9 minimal agar plate in a phage-free working space and the culture was later used for infection with the selected phage. Culture was shaken at 160 rpm and 37° C. until an OD600 nm of 0.6 was reached. E. coli culture was kept on ice until required for infection of eluted phage.

After antigen coating and blocking, blocked phages were transferred to respective wells and incubated for 1 h at RT. To remove unspecific or weakly bound phages, several washing steps with PBST and PBS were performed. In the first panning, standard washing steps were applied (5×PBST quick, 3×PBST for 5 min, 3×PBS quick). Depending on phage output titers, stringency was adapted in the subsequent round by increasing the number and time of the washing steps.

Following antigen-phage incubation and washing, bound phages were eluted by addition of trypsin (250 μl of a 10 μg/ml trypsin solution in PBS, 30 min at 37° C.) to cleave the proteinase-sensitive linker between the antibody fragment and the gIII protein. The phage suspension of each selection was transferred to a pre-warmed E. coli ER2738 culture each and incubated for 45 min in a water bath at 37° C. without shaking. Bacterial cultures were centrifuged, supernatants were removed, and pellets were resuspended in 2xYT medium. The bacteria were plated on LB/Cam agar plates and incubated over night at 37° C. On the next day, bacteria were scraped of the plates with freezing medium (2xYT medium containing 34 μg/ml chloramphenicol (Cam), 1% glucose and 15% glycerol) and aliquots were stored at-80° C. before preparation of phages for the subsequent panning round.

### Solution and Semi-Solution Panning

For solution and semi solution panning, magnetic beads (GE, Sera-Mag Streptavidin-Coated Magnetic Particles, Cat: #30152104010150) were used in combination with biotinylated cPD-L1_hFc (SinoBiological; Cat.: 70110-DO2H-B) to capture the antigen-phage complex or immobilize antigen on the bead surface, respectively. To reduce the selection of phages against the magnetic beads used, Neutravidin-coated beads (GE, Sera-Mag Neutravidin-Coated Magnetic Particles, Cat: #78152104010150) were alternated with the Streptavidin-Coated beads mentioned before.

Before the panning, beads were washed and blocked. For semi-solution panning, phages were also loaded with antigen. To this end, 250 μl beads for each selection were transferred to 2 ml low binding tubes, beads were captured with a magnetic particle separator and storage solution was removed. Then, beads were washed 3 times with PBS, using a magnet to collect beads for removal of washing buffer. Afterwards, beads were blocked for 1 h at RT in blocking solution (100% Chemiblock). In parallel, phages were blocked. To this end, the required volume of phages was mixed with Chemiblock containing 10 μg/ml hulgG for blocking and incubated for at least 1 h at RT. In addition, blocked phages were pre-absorbed on empty magnetic beads to remove sticky phages. To remove biotin-specific and hFc-tag-specific phages prior to panning, phages were also incubated in 96 well plate well coated with 5 g/ml BSA-biotin for 45 minutes and in wells coated with 5 μg/ml hulgG over night at 4° C., respectively.

After blocking of beads and blocking/pre-adsorption of phages, biotinylated antigen was added to the phage solutions and incubated for 1 h at RT rotating. For capture of the phage/antigen complex, the blocking buffer was removed from the beads and the phages were added to allow binding of the biotinylated cPD-L1_hFc for 20 minutes at RT. Subsequently, unspecific phages were removed by washing (3×PBST quick, 3×PBST for 5 min, 3×PBS quick). Washing stringency was adapted from round to round depending on panning outputs. With the last washing step, magnetic beads with the captured antigen-phage complex were transferred into a fresh low binding tube.

For elution of specific phage, 300 μl trypsin was added for 30 min at 37° C., rotating. Subsequently, phage suspensions of each selection were transferred to 20 ml of pre-warmed E. coli ER2738 culture each and incubated for exactly 45 min in a water bath at 37° C. without shaking. Bacterial cultures were centrifuged for 5 min at 4600 rpm at 4° C. and supernatants were discarded. Pellets were resuspended in 600 μl 2× YT medium and plated on large LB/Cam agar plates and incubated over night at 37° C. Next day, bacteria were scraped of the plates with 1-3 mL freezing medium (2xYT medium containing 34 μg/ml Cam, 1% glucose and 15% glycerol) using a sterile Drygalski spatula and aliquots were stored at-80° C. before preparation of phages for the sub-sequent panning round.

Semi-solution panning was performed similar to solution panning with the following modification: Instead of capturing the complex of phages bound to biotinylated target out of solution using Streptavidin or Neutravidin magnetic beads, the respective antigens were immobilized on the beads already prior to blocking of the beads. Thus, the panning mode reflects a selection on a solid phase but allows better orientation of the target and washing conditions compared to a panning where the antigen is coated on the surface of a microtiter plate. Analogous to the solid phase panning, 3 round were performed, each with reduced cPD-L1_hFc concentration and with increased washing stringency.

### Phage Preparation

For each phage preparation, inoculation medium (2xYT medium containing 34 μg/ml Cam and 1% glucose) was inoculated with phagemid containing bacterial suspension or glycerol stock resulting in an OD600 of around 0.2. Cultures were incubated for 60-120 min at 37° C. shaking until an OD600 of around 0.5 to 0.6 was reached. VCSM13 helper phages were added and incubated for 30 min at 37° C. without shaking and then for 30 min at 37° C. shaking at 250 rpm. Subsequently, bacteria were spun down and helper phage containing supernatant was discarded. Phage infected bacteria were resuspended in induction medium (2xYT medium containing 50 μg/ml Carbenicillin, 50 μg/ml Kanamycin (Kan) and 0.2 mM IPTG and incubated for 18-20 h at 22° C., shaking at 200 rpm in a phage shaker. Next day, bacteria were spun down and the supernatant containing the antibody-presenting phages were transferred to new tubes. For phage precipitation, 1/5 volume of ice-cold PEG/NaCl was added to the phage-containing supernatant, mixed and incubated for at least 30 min on ice, gently shaking. Precipitated phages were spun down for at least 30 min at 10000 × g at 4° C. Supernatants were removed quantitatively and phage pellets were resuspended in an adequate volume of PBS. Phages were stored at 4° C. rotating for short periods or frozen at −80° C. for longer storage.

### Subcloning and Generation of Screening Plates

After multiple rounds of panning, polyclonal phage outputs were subcloned into the bacterial Fab expression vector pCaBx. Antibody encoding fragments were removed from the phage display vector using flanking restriction enzymes, isolated using preparative agarose gel electrophoresis (1.0% agarose) and fragments were DNA purified from the gel slice using an appropriate gel extraction kit. The ligation reaction with the insert and pre-cut pCaBx vector and subsequent transformation in chemically competent E. coli BL21 (DE3) was performed according to standard procedures.

Wells of expression plates (Round bottom 96-well plates (e.g., Thermo Fisher, Cat: #262162) were filled with 80 μl/well all-in-one-medium (2xYT medium containing 34 μg/ml Cam, 0.1% Glucose and 0.5 mM IPTG) and were inoculated with single colonies from agar plates of the subcloning procedure. Plates were incubated for 5 h at 37° C. shaking at 600 rpm and afterwards overnight at 22° C. shaking at 600 rpm. To prepare crude bacterial lysates used for screening purposes, so called BEL-lysates, 30 μl/well of lysis buffer (2×BBS containing 2.5 mg/ml lysozyme, 4 mM EDTA and 13 U/ml Benzonase) were added and incubated for 1 h at 22° C. shaking at 600 rpm. Then, 30 μl/well of blocking buffer (1×PBS containing 5% milk powder) were added and incubated at 22° C. for 1 h shaking at 600 rpm. Plates were stored at −20° C. or directly used for screening.

### Screening ELISA and Identification of Unique Clones

For screening, antibody fragment containing lysates (BEL lysates) were tested for binding to antigens immobilized on Maxisorp microtiter plates. Following immobilization of the antigens on the respective surface, plates were washed three times with PBST and subsequently blocked with 5% milk in PBST for 1 h at RT. BEL lysates, control antibodies and negative controls were transferred to plates and incubated for 1 h at RT. Plates were washed 3 times with PBST and subsequently, detection antibody was diluted in PBST 0.5% milk and added to the plates and incubated for 1 h at RT. For the detection, an anti-FLAG antibody (Sinobiological, 109143-MM13) was used. Plates were washed 5 times with PBST, and detection of bound antibodies was performed using the QuantaBlu reagent according to the manufacturer's instruction on a Tecan Genious Reader (excitation filter: 320 nm, emission filter: 430 nm).

Individual clones that were identified by screening in ELISA format were Sanger-sequenced. Therefore, Miniprep cultures from the respective positive hits were inoculated from glycerol stocks of the individual samples. Plasmids were purified with standard protocols and sent for sequencing at an external service provider using primers that cover all CDRs regions that are variable by library design.

Around 65 different antibody variants were identified after screening. Two sequence unique candidates with promising binding properties, referred to as CAN1005001 comprising VL and VH domains according to SEQ ID Nos: 41 and 42 and CAN1005010 comprising VL and VH domains according to SEQ ID Nos: 1 and 2 were characterized in more detail.

### Example 2

Binding ELISA with the initial clones CAN1005001 and CAN1005010 from panning

For characterizing the binding of CAN1005001 and CAN1005010 towards its target cPD-L1, an ELISA setup was used. cPD-L1 fusion proteins were directly immobilized on the plate, commonly in a concentration of 57 nM. The coated plates were washed 3× with PBS-T, followed by blocking with Chemiblocker (Merck-Millipore; 2170) for 1 h at RT. After blocking, dilution series of antibody solutions were added to the plate, incubated for 1 h at RT, followed by washing and by addition of suitable detection antibodies coupled to HRP. Measurement was carried out as described above for the Screening ELISA. The outcome of this experiment is displayed together with Example 3.

### Example 3

Ligand-binding inhibition assays with initial clone CAN1005001 and CAN1005010

To confirm the ability of CAN1005001 and CAN1005010 to block the interaction between canine PD-1 and canine PD-L1, a ligand-binding inhibition assay was setup by using an ELISA format. Immobilized cPD-L1_hFc was presented immobilized on a plate and accessible binding sites were blocked with CAN1005001 and CAN1005010, respectively. The lack of accessible binding sites for PD-1 is then analyzed measuring residual PD-1 binding to PD-L1_hFc.

### ELISA Ligand-Binding Inhibition Assay (LBI)

cPD-L1 fusion proteins were directly immobilized on the plate overnight at 4° C., commonly in a concentration of 57 nM. For LBI ELISA, black 384 well Maxisorp plates were used. The coated plates were washed 3× with PBS-T, followed by blocking with Chemiblocker (Merck-Millipore; 2170) for 1 h at RT. The blocked plates were washed 3× with PBS-T, followed by addition of titration-series of blocking antibodies and incubation for 1 h at RT. Subsequently plates were washed again 3× with PBS-T and PD-1-Biotin (1× biotinylated via Avi-tag; Sinobiological Cat.: 70109-D27H-B) was added and incubated for 1 h at RT, usually in a concentration of 4 μg/ml (206 nM). This concentration of canine PD-1-Biotin was previously identified as optimal by titrating the binding of PD-1-Biotin towards immobilized canine PD-L1_hFc. After incubation with the ligand PD-1, the plate was washed 3× with PBS-T and suitable detection reagent for the biotinylated PD-1, usually Streptavidin-HRP, was added and incubated for 1 h at RT. Subsequently, the plate was washed 5×, followed by addition of Quanta Blue and measurement as described above for the screening ELISA.

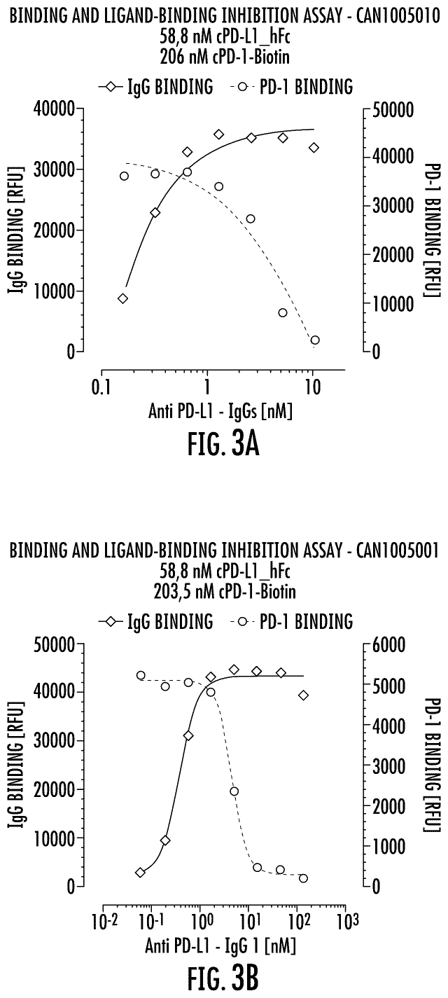

Results are depicted in FIG. 3. CAN1005001 and CAN1005010 show dose dependent binding to cPD-L1. In line with that, increasing amounts of both antibodies efficiently block binding of cPD-1 to immobilized cPD-L1. For CAN1005001, an EC50 value of 0.4 nM and an IC50 value of 4.6 nM was calculated.

### Example 4

Affinity maturation of CAN1005001 and CAN1005010 and screening in Fab format

LBI experiments indicated that a subset of clones, including CAN1005001 and CAN1005010, that can block the interaction between canine PD-1 and canine PD-L1. An affinity maturation strategy was initiated to increase binding strength and potency of respective candidates further.

The maturation approach focused on two sites within these antibodies: Within the heavy chain, the HCDR2 was modified whereas on the light chain, the LCDR3 was removed and replaced by respective maturation modules. Maturation modules are designed in a way that they reflect natural diversity within the respective CDRs, are devoid of critical PTM sites and are highly diversified. For clarification, a “VH-matured clone” contains the original HCDR1, HCDR3, LCDR1, LCDR2 and LCDR3 but altered HCDR2 regions whereas a “VL-matured clone” has a distinct LCDR3 sequence whereas the remaining CDRs are identical to the parental clone. Of note, it is also possible to combine matured chains to generate so called cross-clones with modifications in both VL and VH. There is a common understanding of the correlation between library size and the chance of strong affinity improvement. Thus, the aim was to prepare libraries in the range of more than 1.00E+07 variants.

Prior to affinity maturation, Fab encoding inserts of selected candidates were digested via EcoRI/Ncol and ligated into the phage display vector according to standard procedures. Afterwards, VH or VL stuffer sequences were ligated into these candidates before these were replaced by HCRD2 or LCDR3 maturation modules.

For the preparation of vector backbones for the introduction of maturation modules, stuffers are removed again by restriction digest using BssHII and Mfel for the VH stuffer and BbsI and KpnI or the VL stuffer according to standard procedures.

For VL maturation libraries, maturation modules for the LCDR3 regions are cloned into the vector backbones using BbsI and KpnI as described above. For the VH maturation, similarly HCDR2 modules are introduced by BssHII and Mfel cloning.

For library cloning, transformation is done using highly electrocompetent ER2738 cells and DNA was desalted by precipitation prior to electro transformation. To this end, ligation samples were adjusted to 50 μl with sterile ddH₂O and 1 μl glycogen as well as 500 μl 2-butanol were added and incubated for at least 5 min at RT on a rotator. Then, precipitated DNA was spun down at 4° C. for 30 min at maximum speed in a tabletop centrifuge. Supernatants were discarded and DNA pellets were washed with 500 μl pre-cooled 70% ethanol. Samples were centrifuged again for 15 min at 4° C. and maximum speed, ethanol was removed, and the DNA pellet was air dried for around 15 min. Pellet was resuspended in 5 μL of ddH₂O per ligation approach.

For each library, 2 transformations in ER2738 cells were performed according to the manufacturer's instructions using a BTX electroporator (settings: 25 uF, 20062, 1.6 kV). Immediately after the pulse, cells were transferred to 950 μl pre-warmed recovery medium and incubated for 1 h at 37° C. gently shaking at 200 rpm. A small aliquot of recovered cultures was saved for library size determination. The remaining cultures were spun down at 4600 × g for 10 min, resuspend in 400 μl 2xYT, plated on 2 large LB/Glu/Carb agar plates and incubated over night at 37° C. The next day, bacteria were scraped of the plates using pre-cooled LB medium containing 20% glycerol, aliquots were prepared and stored at-80° C. until subsequent phage preparation.

Library sizes were determined using the Eddy Jet spiral plater and exceeded 2.00E+08 clones per library. QC was done by colony PCR and sequencing of 10 clones per library to confirm diversification and absence of parental clones and a viable cell count was done to evaluate quality of frozen glycerol stocks. Phage preps were essentially performed as described above (Example 1) but were upscaled to reflect the higher number of individual clones within the library and the requirement to cover this larger diversity also at the point of phage production.

Maturation pannings have been performed essentially as described above (Example 1), mainly using solution pannings. In contrast to initial pannings, maturation pannings were performed under more stringent conditions. This was achieved by reducing antigen concentrations during panning, i.e., for the first panning round, target amounts in a lower range as in the third round of initial pannings were used. Also, washing steps were prolonged and increased in numbers. In addition, a koff selection step was included. After the antigen-phage complex was captured using magnetic Streptavidin beads and beads were washed to remove unwanted phages, a 10-fold molar excess of unbiotinylated antigen was added to the wash buffer and incubated overnight. During this step, low affinity antibodies will dissociate from the antigen captured on beads, but rather find another interaction partner in solution than on the bead surface. Thus, only high affinity antibodies shall be recovered. After the koff selection step, supernatants containing lower affinity antibodies were removed, beads washed again with PBS and eluted as described before.

The maturation panning succeeded in identifying 186 clones that showed superior signal than the respective parental clone. This included maturation panning campaigns for other parental clones. For the parental clone CAN1005010, the clones CAN1005010L1 from the LCDR3 maturation campaign (comprising SEQ ID NO: 9) and CAN1005010H2 (comprising SEQ ID NO: 19) from the HCDR2 maturation campaign were selected for further studies. Respectively, for the parental clone CAN1005001, the clones CAN1005001L1 (comprising SEQ ID NO: 47) from the LCDR3 maturation campaign and CAN1005001H3 (comprising SEQ ID NO: 52) from the HCDR2 maturation campaign were selected for further studies.

### Fab-Capture ELISA Screening

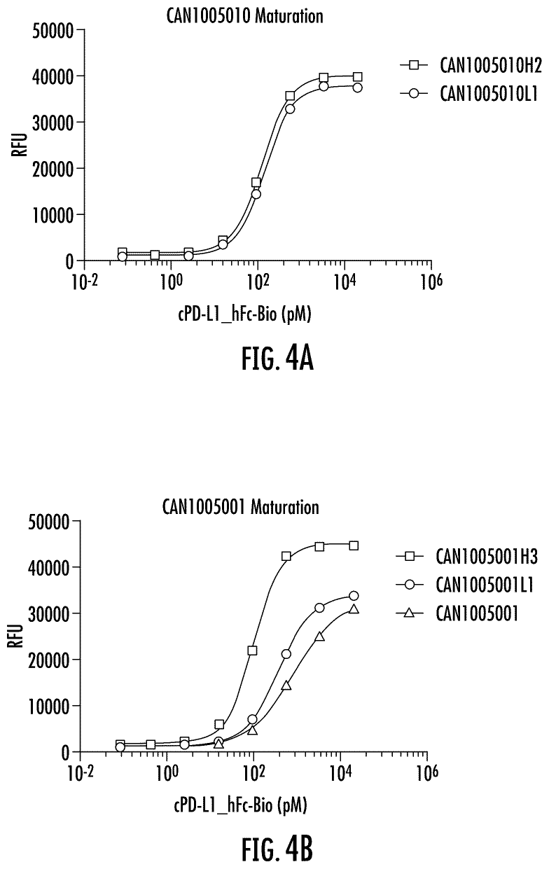

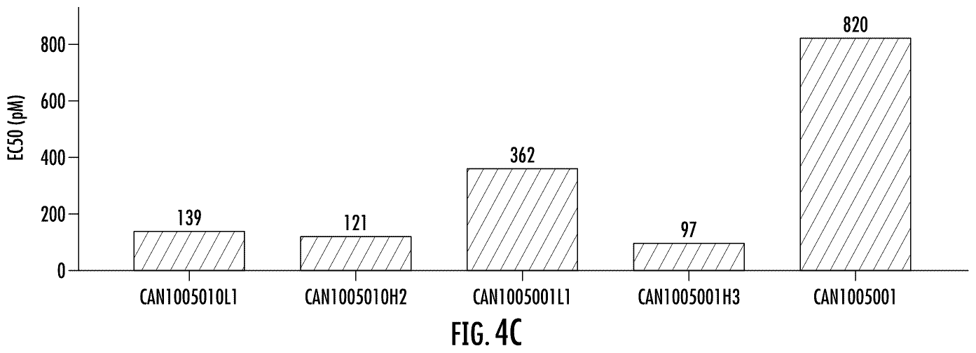

To identify derivatives with a higher affinity, an alternative ELISA setup was used for screening to better normalize BEL expression levels and differentiate Fabs more efficiently with respect to their affinity. In contrast to the previous screening ELISA setup, a Maxisorp plate was coated over night with an anti-canine Fab antibody in a low density of 1 μg/ml. Coated plates were washed 3× with PBS-T, followed by loading with diluted BELs. After the BEL incubation and washing with PBS-T, dilution series of cPD-L1-biotin was added to the plate. Detection was performed with Streptavidin-HRP, followed by more stringent 5× washing with PBS-T and Quanta Blue substrate addition. Measurement was performed as described before (Example 1). Resulting data are shown in FIG. 4.

Compared to parental clones, derivatives from both the heavy and light chain maturation with significantly better binding towards cPD-L1 were identified. This is expressed in EC50 values of 362 pM for CAN1005001L1 and 97 pM for CAN1005001H3, compared to 820 pM for the parental clone CAN1005001. For CAN1005010L1 and CAN1005010H2, EC50 values for 139 PM and 121 pM, respectively, were determined. The parental clone CAN1005010 was not included in the experiment shown.

### Example 5

Cross-cloning of different maturated heavy and light chains derived from CAN1005010 and CAN1005001

To further increase the affinity of CAN1005010 derivates CAN1005010L1 and CAN1005010H2 (comprising SEQ ID NO: 9 and 19) the respective maturated light and heavy chains were combined in a single Fab, that only shares the HCDR3 with CAN1005010 and contains both maturated LCDR3 and HCDR2. In the same manner, the respective maturated light, and heavy chains from the CAN1005001 derivatives and CAN1005001H3 (comprising SEQ ID NO: 47 and 52) were combined in a single Fab.

The resulting clones were termed CAN1005016 and CAN1005019, respectively. Cloning was performed according to standard procedures as described below. This involved initial digest both clones, whereas one served as vector backbone and the other on served as insert. In this case, the LCDR3 maturated clones CAN1005010L1 (comprising SEQ ID NO: 39) and CAN1005001L1 (comprising SEQ ID NO: 65) served as vector backbone. The resulting Fabs were tested in a Fab-capture ELISA as previously reported in Example 4.

### Restriction Digest

5 μg of both vectors was digested with Mfel and Xhol to remove the heavy chain coding region. Restriction digest was carried out at 37° C. for 1 h, followed by inactivation at 80° C. for 20 min. The backbone sample digest further contained FastAP to inhibit vector relegation. For CAN1005010L1, the vector lacking the heavy chain was excised from an agarose gel and purified by standard procedure. The corresponding insert originating from clone CAN1005010H2 was also applied on an agarose gel and the insert was excised and purified by standard procedure.

### Ligation

After restriction digest, the insert was ligated into the vector backbone. A mix of 5:1 insert to vector was prepared and T4 ligase (NEB; Cat.: M0202S) was added according to standard instructions. Ligation was carried out at 16° C. overnight for 16 h. The final ligation product was directly transformed into chemically competent E. coli cells. 10 μl ligation product was mixed with 100 μl of competent cells and incubated for 30 min on ice. Then the cells were placed in a water bath at 42° C. for 10⁻⁴⁵ seconds, depending on type of E. coli cells. After rescue of the transformed cells in fresh 2xYT medium for 1 h, the cells were centrifuged and resuspended in a volume appropriate for plating on LB agar plates containing the respective selection antibiotic of the vector.

### Fab-Capture ELISA Screening

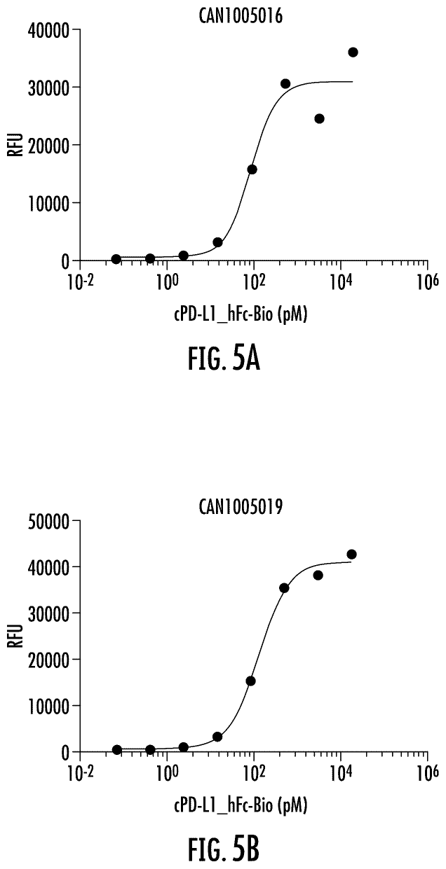

The screening was performed as described in Example 4. Fab binders of CAN1005016 and CAN1005019 were loaded onto plate-bound anti-canine Fab capture antibody, followed by addition of titration series of cPD-L1_hFc-Biotin. Detection was performed with Streptavidin-HRP. The parental clone CAN1005001 was included as a control. Curve fits and EC50 values were analyzed with GraphPad Prism. Results are shown in FIG. 5.

CAN1005016 was generated through combination of CAN1005010L1 and CAN1005010H2, whereas CAN1005019 resulted from combining CAN1005001L1 and CAN1005001H3. For CAN1005016, an EC50 of 87 pM was determined, demonstrating an improvement compared to its parental clones as depicted in FIG. 5. For CAN1005019, an EC50 of 138 pM was determined.

### Example 6

Grating-coupled Interferometry (GCI) measurements to characterize anti-PD-L1 IgGs

To further characterize the kinetic properties of our candidates CAN1005016 and CAN1005019, the K_{D} of our candidates was quantified using waveRAPID in a GCI system according to the procedure described below. The binding kinetics study was performed on the Creoptix® WAVEdelta system using a PAG sensor chip (Creoptix, a Malvern Panalytical brand). All experiments were performed with HBS-EP (Cytiva) as running buffer, at a temperature of 25° C. and sampling rate of 10 Hz. Data acquisition and evaluation were performed using WAVEcontrol software version 4.5.13.

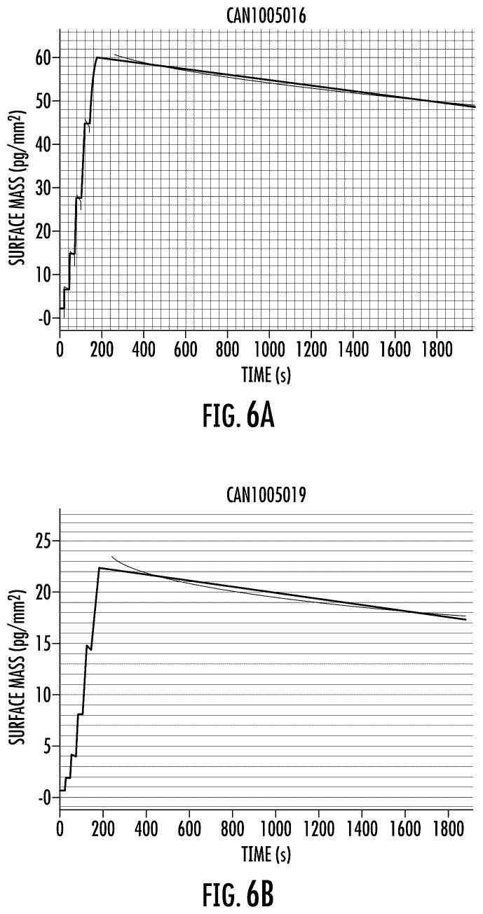

Prior to ligand capturing, the sensor chip was conditioned by a 180 sec injection of 100 mM sodium borate, 1 M NaCl, pH 9 (Xantec). Antibody was diluted to 1 μg/ml in HBS-EP and captured to a density of =300 pg/mm2 on flow channel 2. Flow channel 1 was left blank to serve as a reference surface. For collection of kinetic data, PD-L1-his was diluted to 200 nM in HBS-EP and injected over ligand and reference surfaces in repeated analyte pulses of increasing duration (waveRAPID) at flowrate of 60 μl/min. PD-L1-his was injected for a total duration of 180 sec, followed by measuring complex-dissociation in running buffer for 30 min. Blank injections were performed to enable double referencing of the data and a 0.5% DMSO pulse injection (running buffer with 0.5% DMSO) was used as an analyte concentration modulation calibration curve. Data were corrected (X and Y offset, DMSO calibration, double referencing) and fitted with a simple 1:1 interaction model (global fit) to obtain kinetic parameters. Results are shown in FIG. 6.

A Kp of 0.77 nM was determined for CAN1005016, and a Kp of 1.7 nM was determined for CAN1005019. As comparison, the parental clone CAN1005010H2 has a Kp of 2.8 nM. Thus, cross-cloning yielded a >3-fold improvement in affinity for CAN1005016.

### Example 7

Generating binding profiles of CAN1005016 towards cPD-L1_hFc with ELISA and FACS

For characterizing the binding of CAN1005016 towards its target cPD-L1, an ELISA setup was used. ELISA was performed according to standard procedures as described below with cPD-L1_hFc or cPD-L1_MBP immobilized on the plate directly. Additionally, binding towards HEK293c18 cells transfected with cPD-L1 was tested in flow cytometry (FACS).

### ELISA

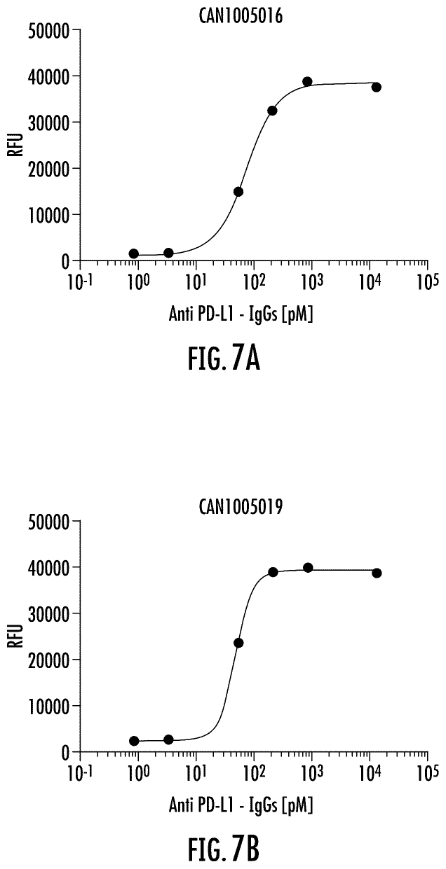

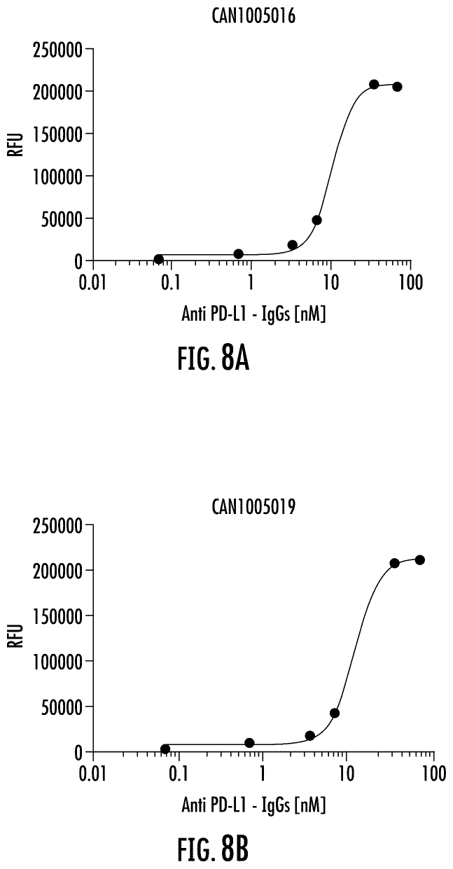

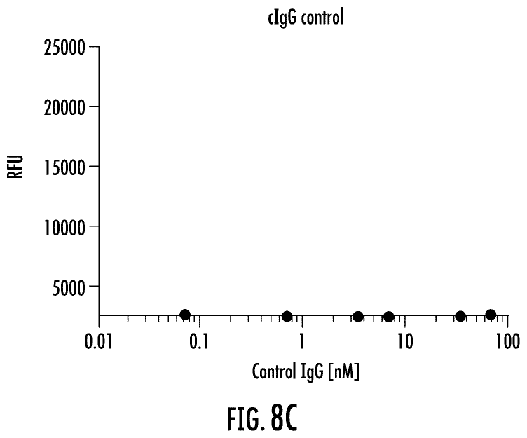

cPD-L1 fusion proteins were directly immobilized on the plate, commonly in a concentration of 57 nM. For binding ELISA, black 384 well Maxisorp plates were used. This was performed overnight at 4° C. The coated plates were washed 3× with PBS-T, followed by blocking with Chemiblocker (Merck-Millipore; 2170) for 1 h at RT. After blocking, dilution series of the respective antibodies were added to the plate usually covering concentrations ranging from the single digit ng/ml to the single-digit ug/ml range. Antibodies were also incubated for 1 h at RT, followed by washing and by addition of suitable detection antibodies coupled to HRP. Measurement was carried out as described above for the Screening ELISA. Results are shown in FIG. 7.

The maturated and cross-cloned IgGs CAN1005016 and CAN1005019 show dose-dependent binding to cPD-L1 with EC50 values of 76 pM and 49 PM, respectively. This is a significant improvement to the 400 pM EC50 value of CAN1005001 IgG measured in Example 3.

### Transfection of HEK293c18 Cells

HEK293c18 cells were seeded in 6 well plates at a density of 1×10⁶ cells/well in DMEM+10% FCS. The next day, the transfection mix was prepared. Transfection was performed with jetPRIME transfection reagents (Polyplus, Cat.: 101000046). Per well, 2 μg of the plasmid encoding for wildtype canine PD-L1 was diluted in 200 μl jetPRIME buffer. The solution was mixed and 4 μl of the transfection reagent was added, followed by mixing and centrifuging of the mixture. Then, the medium of the adherent cells is replaced by fresh medium and the transfection mix is carefully added onto the cells. The plate containing the transfected cells is placed back in the incubator and incubated for 24 h.

The next day, the cells were harvested with PBS+2 mM EDTA (PBS/EDTA) by gentle pipetting, counted, and 2×10⁵ cells/well were transferred to a 96 well V-bottom plate for FACS staining.

### FACS Staining

The cells were washed once by addition of 200 μl per well PBS/EDTA and centrifugation at 1500 rpm for 5 min. The supernatant was carefully decanted by holding the plate upside down above a waste and dried on a paper towel. Then, the cells were stained for dead cells with the dye Zombie violet (Biolegend; Cat.: 423114) diluted 1:500 in PBS/EDTA for 15 minutes at 4° C. in the dark. Subsequently, cells were washed with FACS Buffer (PBS/EDTA+2% FCS) as described above. Titration series of CAN1005016 and CAN1005019 usually ranging from 10 ng/ml to 10 μg/ml were prepared diluted in FACS buffer and added to the respective wells for a 30-minute incubation at 4° C. in the dark. After incubation, the cells were washed 3× with FACS buffer and the detection antibody goat-anti-canine-IgG FITC (ThermoFisher; Cat.: #A18764) was added and incubated for 30 minutes at 4° C. in the dark. Finally, the cells were washed again 3× and then resuspended in 200 μl FACS buffer and measured directly. Results are shown in FIG. 8.

Both candidates recognize cell-bound cPD-L1 as demonstrated by FACS on HEK293c 18 cells transfected with the target protein. EC50 values of 10.5 nM were determined for CAN1005016 and of 11.8 nM for CAN1005019. No staining was observed for an irrelevant control antibody.

### Example 8

Ligand-binding inhibition assay with CAN1005016 and CAN1005019 in ELISA and BLI setup

### ELISA Ligand-Binding Inhibition Assay (Lbia)

To confirm the ability of CAN1005016 and CAN1005019 to block the interaction between canine PD-1 and canine PD-L1, a ligand-binding inhibition assay was setup by using an ELISA format. cPD-L1_hFc was immobilized on a plate and accessible binding sites were blocked with the respective candidates. The lack of accessible binding sites for PD-1 was analyzed measuring residual PD-1 binding to PD-L1_hFc.

cPD-L1 fusion proteins were directly immobilized on the plate overnight at 4° C., commonly in a concentration of 57 nM. For Ibia ELISA, black 384 well Maxisorp plates were used. The coated plates were washed 3× with PBS-T, followed by blocking with Chemiblocker (Merck-Millipore; 2170) for 1 h at RT. The blocked plates were washed 3× with PBS-T, followed by addition of titration-series of blocking antibodies and incubation for 1 h at RT. Subsequently plates were washed again 3× with PBS-T and PD-1-Biotin (1× biotinylated via Avi-tag; Sinobiological Cat.: 70109-D27H-B) was added and incubated for 1 h at RT, usually in a concentration of 4 μg/ml (206 nM). This concentration of canine PD-1-Biotin was previously identified as optimal by titrating the binding of PD-1-Biotin towards immobilized canine PD-L1_hFc. After incubation with the ligand PD-1, the plate was washed 3× with PBS-T and suitable detection reagent for the biotinylated PD-1, usually Streptavidin-HRP, was added and incubated for 1 h at RT. Subsequently, the plate was washed 5×, followed by addition of Quanta Blue and measurement as described above for the screening ELISA. Results are shown in FIG. 9.

Both candidates can efficiently block the interaction between cPD-L1 and cPD-1. IC50 values of 0.95 nM were determined for CAN1005016 and of 1.32 nM for CAN1005019.

### Biolayer-Interferometry (BLI)-Based Ligand-Binding Inhibition Assay (CAN1005016 Only)

A reverse setup was tested in Biolayer-Interferometry. Here, PD-1 was immobilized on a sensor and a mixture of cPD-L1_hFc and anti-PD-L1 antibody CAN1005016 competed for binding to cPD-L1. All reagents were diluted in Kinetics Buffer (Sartorius; 18-1105).

Biotinylated canine PD-1 (cPD-1-Bio) was immobilized on a Streptavidin Sensor (Sartorius; Cat.: 18-5019). Therefore, after a 30 sec baseline phase, 5 μg/ml cPD-1-Bio solution was immobilized for 45 seconds until a signal of 1-2 nm was reached. This was followed by 30 sec baseline in Kinetics buffer. Subsequently, pre-mixed solutions of 100 nM cPD-L1 with different concentrations of CAN1005016 (10 nM⁻¹⁰⁰ nM) were associated to the loaded sensor for 150 sec. After association, 120 sec of dissociation were recorded. Measurement was carried out on a BLItz system (Pall Fortebio)

Pre-incubation of cPD-L1 with CAN1005016 at stoichiometric amounts leads to blocking of interaction sites towards cPD-1 and lack of binding of cPD-L1 to the immobilized cPD-1, confirming results from ELISA-based ligand-binding inhibition assays. When using non-stoichiometric amounts of CAN1005016 (1:10), the binding of PD-L1 to the immobilized cPD-1 is partially blocked, as demonstrated in an intermediate signal for cPD-L1 binding. Stochiometric pre-incubation with an irrelevant canine control IgG only has a minor effect on cPD-L1 binding to cPD-1.

### Example 9

Binding of CAN1005016 and CAN1005016 to canine cell lines expressing PD-L1

To verify binding of the antibody candidates to endogenous canine PD-L1, CAN1005016 and CAN1005019 were used to stain for PD-L1 on an IFN-g treated canine cell line. The canine squamous cell carcinoma cell line SCC1 is reported to express canine PD-L1 after stimulation with IFN-g (Pantelyushin et al. 2021).

Treatment of SCC1 Cells with IFN-g

SCC1 cells were cultured in DMEM high glucose medium (Sigma-Aldrich, Cat.: D6429) supplemented with 15% FCS (PAN Biotech, Cat.: P30-3302), non-essential amino acids, and Penicillin-Streptomycin (Thermo-Fisher, Cat.: 15140122). The cells were detached with Trypsin (Thermo Fisher, Cat.: 12604013), washed and resuspended in fresh medium. The cells were counted and 1×10⁶ cells per well were seeded in 6 well plates. The cells were incubated for 3 hours in an incubator at 37° C., 5% CO2, followed by treatment for 2 days with 10 ng/ml IFN-g. Control cells without IFN-g treatment were cultured alongside.

FACS staining of treated SCC1 cells with CAN1005016 and CAN1005019

After treatment, the cells were detached with trypsin (Thermo Fisher, Cat.: 12604013), filtered through a 70 μm or 100 μm cell strainer, and washed 2× with PBS-EDTA. From this point on, the cells were kept cool at 4° C. The cells were stained with Zombie violet live/dead staining (Biolegend, Cat.: 423114), and equal cell numbers were distributed in a 96 well plate for staining. Treated cells and control cells were stained for 1 hour separately with different concentrations of biotinylated CAN1005016, CAN1005019, and control IgG, diluted in FACS Buffer (PBS-EDTA+2% FCS). Then the cells were washed 3× with FACS Buffer by centrifugation at 1500 rpm at 4° C. As detection antibody, Streptavidin-Allophycocyanin (Biolegend, Cat.: 405207) was used and stained for 30 minutes. After incubation, the cells were washed 3× again as described before and resuspended in 200 μl FACS buffer before measuring at a Beckman Coulter Cytoflex LX.

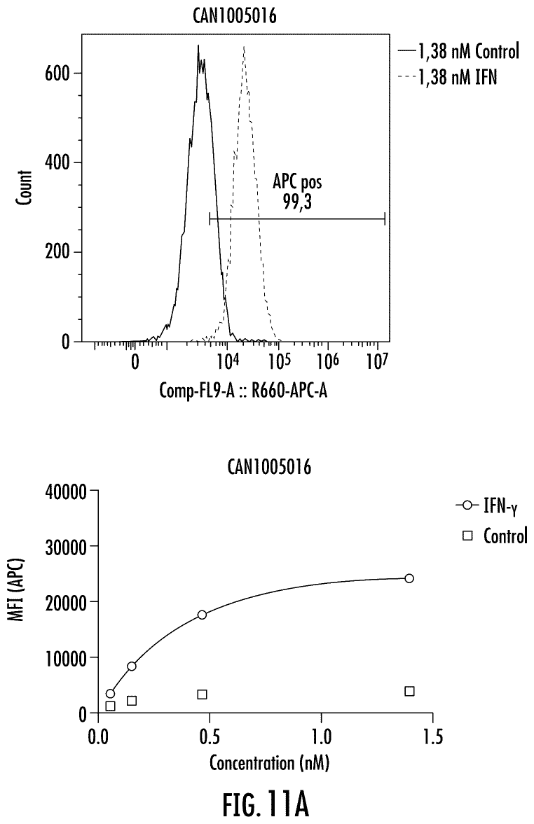

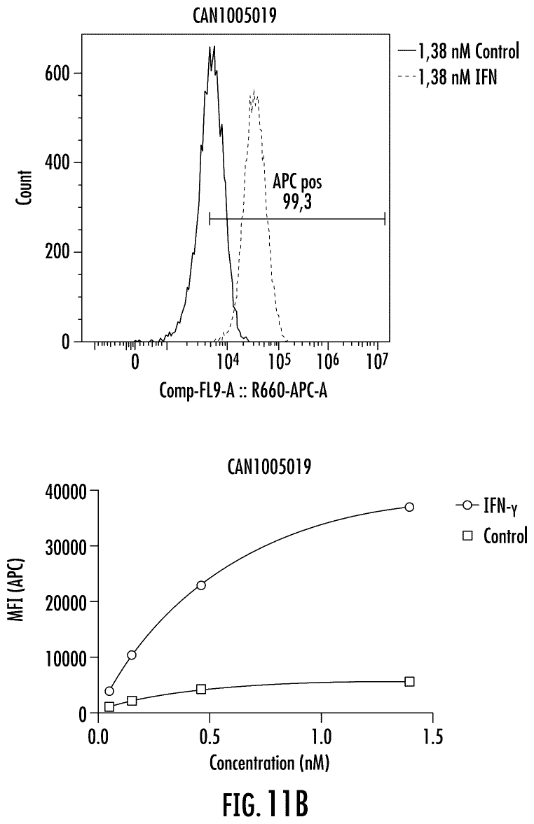

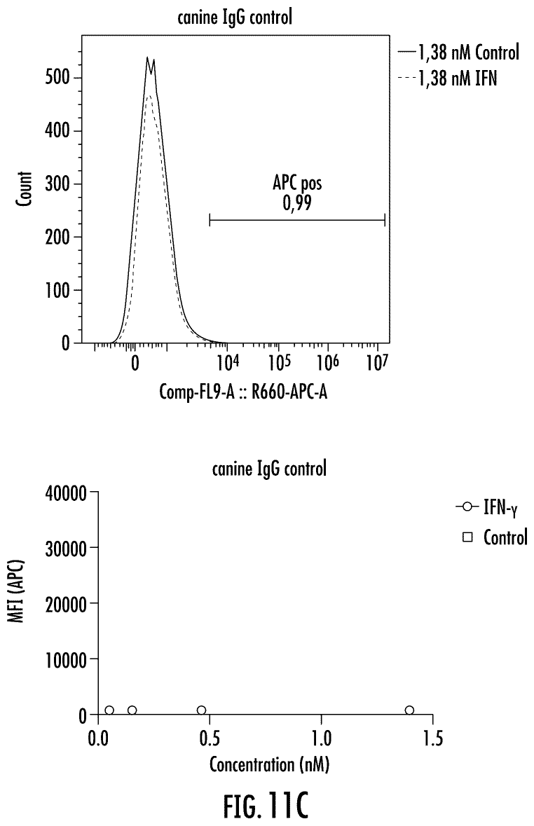

CAN1005016 and CAN1005019 bind to PD-L1 expressing canine cancer cell lines (FIGS. 11A-11C). The treatment with IFN-g, triggering physiological upregulation of canine PD-L1, leads to improved binding of the anti-PD-L1 IgGs to SCC1 cells. This confirms the ability of CAN1005016 and CAN1005019 to recognize cPD-L1 in a cellular context.

### Example 10

Expression, purification, and thermal stability of CAN1005016 and CAN1005019

The plasmid encoding for CAN1005016 and CAN1005019 were transfected separately into suspension HEK293-Freestyle (HEK293-F) cells. Purification of the IgGs was performed by Protein A affinity chromatography followed by Size exclusion chromatography (SEC). The details are described below. The structural properties of CAN1005016 and CAN1005019 were further characterized by Differential scanning fluorimetry (nanoDSF).

### Transfection of HEK293-F Cells and 1gG Expression

HEK293-F cells were cultured in Freestyle medium (ThermoFisher; Cat.: 12338026) in an incubator at 37° C., 5% CO2, and under constant rotation at 135 rpm. Approximately 18-24 hours prior to transfection, HEK293-F cells were counted and seeded at a density of 1.5×10⁶ cells/ml in an appropriate volume for expression, usually 25 ml to 100 ml Freestyle medium. The next day, the transfection mix was prepared according to the volume of cells. In general, 3 ug plasmid and 9 ug PEI per ml culture volume were used and scaled accordingly. The transfection mix was added dropwise to the cells while stirring the flask. The transfected cells were incubated for 5-6 hours before the culture was diluted with fresh pre-warmed Freestyle medium containing 5 mM valproic acid (VPA) as additive for a final concentration of 2.5 mM VPA. Expression was usually carried out for 5-7 days and supernatants were harvested by centrifugation and filtration.

### Purification of CAN1005016 and CAN1005019

Both IgGs were purified via protein-A affinity chromatography, using standard buffers for binding and elution. Eluted IgGs are concentrated to volume <5 ml using a centrifugal concentrator (Amicon, 100 kDa, Cat.: ACS510024) and applied to a Superdex 200 16/600 pg size exclusion column, operated according to standard procedures. The main peak was collected, analyzed via SDS-PAGE and pooled. Purifications from transient, non-optimized expression cultures normally yield 10⁻²⁵ mg/L highly pure IgG.

nanoDSF and DLS measurement

The FORMOscreen® preformulation study was performed on the Prometheus PANTA system for parallel DLS and nanoDSF measurements (Nanotemper Technologies GmbH). All experiments were performed with high sensitivity capillaries (Nanotemper Technologies GmbH) in duplicates and data analysis was performed with merged datasets per tested condition. Data analysis was and evaluation was performed using PANTA Analysis software version 1.4.4.

The protein stock (concentration=29.04 uM) was centrifuged for 1 h at 21.000 g at 4° C. The supernatant was separated, and the concentration was measured again (concentration after centrifugation=28.72 M). For each FORMOscreen® condition, 1.32 μl of the protein stock supernatant was mixed with 4 μl 5×FORMOscreen buffer stock and 14.68 μl ddH₂₀ to reach a final volume of 20 μl with 1.9 M final protein concentration per 1×FORMOscreen® buffer condition.

The samples were prepared in a 384 well AURORA microplate and incubated for 7 days at 40° C. prior to the measurement. Next, the DLS measurements were performed with 10 acquisitions per capillary and 10 us measurement time per acquisition. The nanoDSF measurements were performed with a heating ramp of 1° C./min from 20° C. to 95° C.

TABLE 3
DLS measurement results

| | | Ratio [symbol] | | | | | | Turbidity [symbol] | |
|---|---|---|---|---|---|---|---|---|---|
| | | ON (° C.) | | IP #1 (° C.) | | IP #2 (° C.) | | ON (° C.) | |
| target [symbol] | buffer [symbol] | ø [symbol] | σ [symbol] | ø [symbol] | σ [symbol] | ø [symbol] | σ [symbol] | ø [symbol] | σ [symbol] |
| CAN1005016 | 1x PBS pH 7.4 | 60.38 | 0.16 | 65.30 | 0.06 | 73.31 | 0.07 | 64.49 | 0.16 |
| CAN1005019 | 1x PBS pH 7.4 | 59.06 | 0.14 | 63.67 | 0.05 | | | 61.12 | 1.04 |

### Example 11

Manufacture of CAN1005016

CAN1005016 used in this in-patient study was produced in genetically engineered CHO cells according to standard bioreactor fermentation techniques. Supernatant production was performed using a high expressing stable cell pool in a suitable fed-batch process scale (5L). For harvest of supernatant, cells were removed from cell suspension by centrifugation. The mAb was purified from the supernatant using a one-step Protein A affinity chromatography, formulated and sterile filtered. The formulation has been developed to stabilize the monoclonal antibody for storage at low temperatures and to allow freeze/thawing of the antibody. It consisted of the antibody formulated in a histidine buffer pH 5.9, Trehalose was added as a cryoprotectant, Tween@80 to prevent aggregation.

### Example 12

In vivo testing of CAN10005016

To investigate the efficacy of the anti-canine PD-L1 antibody, CAN10005016 was tested in a female dog (14 years, mixed breed, 14.3 kg) with stage IV oral melanoma and distant metastases in the lung. At the time of enrollment, the size of the primary tumor lesion in the oral cavity was 50×49 mm. CAN1005016 was administered intravenously over 30 min at the dose of 5 mg/kg body weight on a biweekly basis four times, then once monthly. Safety was assessed at each scheduled treatment session using the Veterinary Co-operative Oncology Group (VCOG) criteria (Veterinary Cooperative Oncology Group. Vet Comp Oncol. 2016; 14:417-46.) To monitor systemic toxicity, at each antibody administration 5 ml of blood were taken to obtain CBC, serum biochemistry and thyroid profile. No adverse events were reported throughout the study period. For antitumor response, the dog was evaluated on a monthly basis by means of physical examination, thoracic radiographs and abdominal ultrasound, the tumor size was measured by a caliper and recorded. Antitumor response was classified according to RECIST criteria (Nguyen S M et al., Vet Comp Oncol. 2015; 13:176-83.)

The patient obtained complete remission in the oral cavity after five doses. After the seventh dose, this dog was still in complete remission in the oral cavity. Treatment was extended, reaching a total of 10 doses. 229 days after enrollment, the patient was alive and doing clinically well but recurrent maxillary melanoma and progressive lung metastasis were observed.

Across the veterinary literature, it has been documented that dogs with stage IV melanoma have a poor prognosis, with median survival times ranging from 60 to 80 days when treated with surgery, radiation therapy and/or chemotherapy (Bergman, P. J. Canine oral melanoma. Clin. Tech. Small Anim. Pract. 2007; 22:55, Kawabe M, et al., J Am Vet Med Assoc. 2015 247:1146, Tuohy J L, et al., J Am Vet Med Assoc. 2014; 245:1266.) At the time of preparation of this patent application, the patient was still alive, with overall survival time >300 days.

These results indicate that anti-PD-L1 treatment can be considered as a promising treatment option to treat diseases with high medical need such as malignant melanoma.

### REFERENCES

Ahmadzadeh, Mojgan; Johnson, Laura A.; Heemskerk, Bianca; Wunderlich, John R.; Dudley, Mark E.; White, Donald E.; Rosenberg, Steven A. (2009): Tumor antigen-specific CD8 T cells infiltrating the tumor express high levels of PD-1 and are functionally impaired. In Blood 114 (8), pp. 1537-1544. DOI: 10.1182/blood-2008-12-195792.
Ai, Leilei; Chen, Jian; Yan, Hao; He, Qiaojun; Luo, Peihua; Xu, Zhifei; Yang, Xiaochun (2020): Research Status and Outlook of PD-1/PD-L1 Inhibitors for Cancer Therapy. In Drug design, development and therapy 14, pp. 3625-3649. DOI: 10.2147/DDDT.S267433.
Aresu, Luca; Marconato, Laura; Martini, Valeria; Fanelli, Antonella; Licenziato, Luca; Foiani, Greta et al. (2021): Prognostic Value of PD-L1, PD-1 and CD8A in Canine Diffuse Large B-Cell Lymphoma Detected by RNAscope. In Veterinary sciences 8 (7). DOI: 10.3390/vetsci8070120.
Ariyarathna, Harsha; Thomson, Neroli A.; Aberdein, Danielle; Perrott, Matthew R.; Munday, John S. (2020): Increased programmed death ligand (PD-L1) and cytotoxic T-lymphocyte antigen-4 (CTLA-4) expression is associated with metastasis and poor prognosis in malignant canine mammary gland tumours. In Veterinary immunology and immunopathology 230, p. 110142. DOI: 10.1016/j.vetimm.2020.110142.
Bellucci, Roberto; Martin, Allison; Bommarito, Davide; Wang, Kathy; Hansen, Steen H.; Freeman, Gordon J.; Ritz, Jerome (2015): Interferon-y-induced activation of JAKI and JAK2 suppresses tumor cell susceptibility to NK cells through upregulation of PD-L1 expression. In Oncoimmunology 4 (6), e1008824. DOI: 10.1080/2162402X.2015.1008824.
Cascio, Matthew J.; Whitley, Elizabeth M.; Sahay, Bikash; Cortes-Hinojosa, Galaxia; Chang, Lung-Ji; Cowart, Jonathan et al. (2021): Canine osteosarcoma checkpoint expression correlates with metastasis and T-cell infiltrate. In Veterinary immunology and immunopathology 232, p. 110169. DOI: 10.1016/j.vetimm.2020.110169.
Gearing, D. P.; Huebner, M.; Virtue, E. R.; Knight, K.; Hansen, P.; Lascelles, B. D. X. et al. (2016): In Vitro and In Vivo Characterization of a Fully Felinized Therapeutic Anti-Nerve Growth Factor Monoclonal Antibody for the Treatment of Pain in Cats. In Journal of veterinary internal medicine 30 (4), pp. 1129-1137. DOI: 10.1111/jvim.13985.
Gearing, David P.; Virtue, Elena R.; Gearing, Robert P.; Drew, Alexander C. (2013): A fully caninized anti-NGF monoclonal antibody for pain relief in dogs. In BMC veterinary research 9, p. 226. DOI: 10.1186/1746-6148-9-226.
Han, Yanyan; Liu, Dandan; Li, Lianhong (2020): PD-1/PD-L1 pathway: current researches in cancer. In American Journal of Cancer Research 10 (3), pp. 727-742.
Hartley, G.; Elmslie, R.; Dow, S.; Guth, A. (2018): Checkpoint molecule expression by B and T cell lymphomas in dogs. In Veterinary and comparative oncology 16 (3), pp. 352-360. DOI: 10.1111/vco.12386.
Hartley, G.; Faulhaber, E.; Caldwell, A.; Coy, J.; Kurihara, J.; Guth, A. et al. (2017): Immune regulation of canine tumour and macrophage PD-L1 expression. In Veterinary and comparative oncology 15 (2), pp. 534 549. DOI: 10.1111/vco. 12197.
Igase, Masaya; Nemoto, Yuki; Itamoto, Kazuhito; Tani, Kenji; Nakaichi, Munekazu; Sakurai, Masashi et al. (2020): A pilot clinical study of the therapeutic antibody against canine PD-1 for advanced spontaneous cancers in dogs. In Scientific reports 10 (1), p. 18311. DOI: 10.1038/s41598-020-75533-4.
Iwai, Yoshiko; Hamanishi, Junzo; Chamoto, Kenji; Honjo, Tasuku (2017): Cancer immunotherapies targeting the PD-1 signalling pathway. In Journal of biomedical science 24 (1), p. 26. DOI: 10.1186/s12929-017-0329-9
Maekawa, Naoya; Konnai, Satoru; Ikebuchi, Ryoyo; Okagawa, Tomohiro; Adachi, Mami; Takagi, Satoshi et al. (2014): Expression of PD-L1 on canine tumor cells and enhancement of IFN-γ production from tumor-infiltrating cells by PD-L1 blockade. In PLOS ONE 9 (6), e98415. DOI: 10.1371/journal.pone.0098415.
Maekawa, Naoya; Konnai, Satoru; Nishimura, Maki; Kagawa, Yumiko; Takagi, Satoshi; Hosoya, Kenji et al. (2021): PD-L1 immunohistochemistry for canine cancers and clinical benefit of anti-PD-L1 antibody in dogs with pulmonary metastatic oral malignant melanoma. In NPJ precision oncology 5 (1), p. 10. DOI: 10.1038/s41698-021-00147-6.
Maekawa, Naoya; Konnai, Satoru; Okagawa, Tomohiro; Nishimori, Asami; Ikebuchi, Ryoyo; Izumi, Yusuke et al. (2016): Immunohistochemical Analysis of PD-L1 Expression in Canine Malignant Cancers and PD-1 Expression on Lymphocytes in Canine Oral Melanoma. In PLOS ONE 11 (6), e0157176. DOI: 10.1371/journal.pone.0157176.
Maekawa, Naoya; Konnai, Satoru; Takagi, Satoshi; Kagawa, Yumiko; Okagawa, Tomohiro; Nishimori, Asami et al. (2017): A canine chimeric monoclonal antibody targeting PD-L1 and its clinical efficacy in canine oral malignant melanoma or undifferentiated sarcoma. In Scientific reports 7 (1), p. 8951. DOI: 10.1038/s41598-017-09444-2.
Mason, Nicola J.; Chester, Nicholas; Xiong, Ailian; Rotolo, Antonia; Wu, Ying; Yoshimoto, Sho et al. (2021): Development of a fully canine anti-canine CTLA4 monoclonal antibody for comparative translational research in dogs with spontaneous tumors. In mAbs 13 (1), p. 2004638. DOI: 10.1080/19420862.2021.2004638.
Ohaegbulam, Kim C.; Assal, Amer; Lazar-Molnar, Eszter; Yao, Yu; Zang, Xingxing (2015): Human cancer immunotherapy with antibodies to the PD-1 and PD-L1 pathway. In Trends in molecular medicine 21 (1), pp. 24-33. DOI: 10.1016/j.molmed.2014.10.009.
Pantelyushin, Stanislav; Ranninger, Elisabeth; Guerrera, Diego; Hutter, Gregor; Maake, Caroline; Markkanen, Enni et al. (2021): Cross-Reactivity and Functionality of Approved Human Immune Checkpoint Blockers in Dogs. In: Cancers 13 (4). DOI: 10.3390/cancers13040785.
Sanmamed, Miguel F.; Chen, Lieping (2014): Inducible expression of B7-H1 (PD-L1) and its selective role in tumor site immune modulation. In Cancer journal (Sudbury, Mass.) 20 (4), pp. 256-261. DOI: 10.1097/PPO.0000000000000061.
Takeuchi, Hiroto; Konnai, Satoru; Maekawa, Naoya; Minato, Erina; Ichikawa, Yoshiki; Kobayashi, Atsushi et al. (2020): Expression Analysis of Canine CMTM6 and CMTM4 as Potential Regulators of the PD-L1 Protein in Canine Cancers. In Frontiers in veterinary science 7, Article 330, p. 330. DOI: 10.3389/fvets.2020.00330.
Tang, Haidong; Liang, Yong; Anders, Robert A.; Taube, Janis M.; Qiu, Xiangyan; Mulgaonkar, Aditi et al. (2018): PD-L1 on host cells is essential for PD-L1 blockade-mediated tumor regression. In The Journal of clinical investigation 128 (2), pp. 580-588. DOI: 10.1172/JCI96061.

1. An antibody or antibody fragment that specifically binds to Programmed Death Ligand 1 (PD-L1) comprising:
a. a Variable Light (VL) chain comprising:
i. a VL Complementarity Determining Region 1 (LCDR1) comprising SEQ ID No.: 3,
ii. an LCDR2 region comprising SEQ ID No.: 4, and
iii. an LCDR3 region comprising either SEQ ID No.: 18 or SEQ ID No.: 49; and/or;
b. a Variable Heavy (VH) chain comprising:
i. a VH Complementarity Determining Region 1 (HCDR1) region comprising either SEQ ID No.: 6 or SEQ ID No.: 44,
ii. an HCDR2 region comprising either SEQ ID No.: 30 or SEQ ID No.: 60, and;
iii. an HCDR3 region comprising either SEQ ID No.: 8 or SEQ ID No.: 46.

2. The An antibody or antibody fragment that specifically binds to PD-L1, comprising:
a. a Variable Light (VL) chain comprising:
i. an LCDR1 comprising SEQ ID No.: 3;
ii. an LCDR2 region comprising SEQ ID No.: 4, and
iii. an LCDR3 region having comprising an amino acid sequence selected from the group consisting of: SEQ ID No.: 5, 9, 10, 11, 12, 13, 14, 15, 16, 17, 43, 47, 48; and
b. a Variable Heavy (VH) chain comprising:
i. an HCDR1 comprising either SEQ ID No.: 6 or SEQ ID No.: 44
ii. an HCDR2 region having an amino acid sequence selected from SEQ ID No.:
7, 19, 20, 21, 22, 23, 24, 25, 26, 27, 28, 29, 45, 50, 51, 52, 53, 54, 55, 56, 57, 58, 59, and;
iii. an HCDR3 region comprising either SEQ ID No.: 8 or SEQ ID No.: 46.

3. The antibody or antibody fragment according to claim 2, comprising: a variable light chain comprising a LCDR3 region according to SEQ ID No.: 18 combined with a variable heavy chain comprising a HCDR2 region according to SEQ ID No.: 30, or comprising a variable light chain comprising a LCDR3 region according to SEQ ID No.: 49 combined with a variable heavy chain comprising a HCDR2 region according to SEQ ID No.: 60.

4. The antibody or antibody fragment according to either claim 1 or claim 2 comprising:
a. a light chain variable domain framework sequences having an amino acid sequence selected from one or more of SEQ ID No.: 31, 32, 33, 34; and/or;
b. a heavy chain variable domain framework sequences having an amino acid sequence selected from one or more of SEQ ID No.: 35, 36, 37, 38 or;
a heavy chain variable domain framework sequences having an amino acid sequence selected from one or more of SEQ ID No.: 61, 62, 63, 64.

5. (canceled)

6. (canceled)

7. The antibody or antibody fragment of claim 2 wherein the antibody or antibody fragment specifically binds to canine PD-L1, optionally wherein said antibody or antibody fragment cross-competes with an antibody fragment according to any of the preceding claims, and wherein optionally the antibody or antibody fragment interferes with or blocks the interaction between canine PD-L1 and canine PD-1.

8. The antibody or antibody fragment according to claim 2, wherein the antibody or antibody fragment specifically binds to canine PD-L1 with a dissociation constant (Kd) of less than about 20 nM, preferably less than about 10 nM, more preferably less than about 5 nM, most preferably less than about 2 nM, and/or wherein the antibody or antibody fragment binds to canine PD-L1 with an Kon rate of at least about 2× 10⁴ [M⁻¹s⁻¹], preferably least about 5× 10⁴ [M 1s⁻¹], and/or with a Koff rate lower than about 1× 10⁻³ [s⁻¹], preferably lower than about 5× 10⁴ [s^{-1].}

9. The antibody or antibody fragment according to claim 2 wherein the antibody is an antibody fragment selected from Fv, scFv, Fab, Fab′, F (ab′) 2, Fab′-SH, VHH.

10. The antibody or antibody fragment according to claim 2, comprising an Fc domain or fragment thereof selected from IgG isotype IgG-A, IgG-B, IgG-C, or IgG-D, most preferably from IgG isotype IgG-B.

11. The antibody or antibody fragment according to claim 10, wherein the Fc fragment comprises at least one substitution of an amino acid at a position selected from at least one of amino acid position 235, 239, 270, or 331, relative to the wild type Fc fragment, preferably selected from at least one of L235, S239, D270, and/or P331, more preferably, wherein the Fc fragment comprises the mutations L235A, S239A, D270A, and P331G, relative to the wild type Fc fragment.

12. (canceled)

13. An antibody or antibody fragment according to one of the preceding claims, wherein said antibody or antibody fragment is canine or caninized, optionally a recombinant canine antibody or antibody fragment, optionally an isolated antibody or antibody fragment.

14. (canceled)

15. A polynucleotide or a plurality of polynucleotides encoding the antibody or antibody fragment according to claim 2.

16. The antibody or antibody fragment of claim 2 comprising:
a. the Variable Light (VL) chain comprising:
i. an LCDR1 comprising an amino acid sequence comprising SEQ ID No.: 3;
ii. an LCDR2 comprising an amino acid sequence comprising SEQ ID No.: 4;
iii. an LCDR3 comprising an amino acid sequence comprising SEQ ID No.: 9; and
b. a Variable Heavy (VH) chain comprising:
i. an HCDR1 comprising SEQ ID No.: 6
ii. an HCDR2 comprising an amino acid sequence comprising SEQ ID No.: 19 and;
iii. an HCDR3 region comprising an amino acid sequence comprising SEQ ID No.: 8.

17. The antibody or antibody fragment of claim 2 comprising:
a. the Variable Light (VL) chain comprising:
i. an LCDR1 comprising an amino acid sequence comprising SEQ ID No.: 3;
ii. an LCDR2 comprising an amino acid sequence comprising SEQ ID No.: 4;
iii. an LCDR3 comprising an amino acid sequence comprising SEQ ID No.: 47; and
b. a Variable Heavy (VH) chain comprising:
i. an HCDR1 comprising an amino acid sequence comprising SEQ ID No.: 44
ii. an HCDR2 comprising an amino acid sequence comprising SEQ ID No.: 45 and;
iii. an HCDR3 region comprising an amino acid sequence comprising SEQ ID No.: 46.

18. The antibody or antibody fragment of claim 16 wherein the variable light chain comprises an amino acid sequence comprising SEQ ID NO. 72, and a variable heavy chain comprises an amino acid sequence comprising SEQ ID NO. 73.

19. The antibody or antibody fragment of claim 17 wherein the variable light chain comprises an amino acid sequence comprising SEQ ID NO. 74, and a variable heavy chain comprising an amino acid sequence comprising SEQ ID NO. 75.

20. A vector comprising the polynucleotides of claim 15.

21. A host cell comprising the polynucleotides of claim 15 or the vector of claim 20.

22. A method of producing an antibody or antigen fragment wherein the host cell of claim 21 is grown under conditions that result in production of the antibody and is followed by the isolation of the antibody from the host cell or the culture medium of the host cell.

23. A pharmaceutical composition comprising a therapeutically effective amount of the antibody or antibody fragment of claim 2, further comprising a pharmaceutically acceptable carrier.

24. A method of treating a subject for a PD-L1 disorder comprising administering to the subject a therapeutically effective amount of the pharmaceutical composition of claim 22

25. The method of claim 24 wherein the PD-L1 disorder comprises cancer.
